(19) United States
(12) Patent Application Publication
(10) Pub. No.: US 2023/0187068 A1
(43) Pub. Date: Jun. 15, 2023

(54) METHODS, APPARATUSES AND COMPUTER PROGRAM PRODUCTS FOR GENERATING PREDICTED MEMBER QUERY VERTICES IN A HEALTHCARE GRAPH DATA OBJECT

(71) Applicant: Optum, Inc., Minnetonka, MN (US)

(72) Inventors: Gregory J. BOSS, Saginaw, MI (US); Ramprasad Anandam GADDAM, Mumbai (IN); Adam RUSSELL, Arlington, MA (US); Vijay S. NORI, Roswell, GA (US)

(21) Appl. No.: 17/643,673

(22) Filed: Dec. 10, 2021

(51) Int. Cl. G16H 50/20; G16H 10/60; G06F 40/40

(52) U.S. Cl. CPC G16H 50/20; G16H 10/60; G06F 40/40

(57) ABSTRACT

Methods, apparatuses, systems, computing devices, and/or the like are provided. An example method may include generating edges connecting attribute vertices to a member vertex in a healthcare graph data object, determining, using at least one graph-based machine learning model and based at least in part on the attribute vertices, historical member vertices from the healthcare graph data object, determining, using the at least one graph-based machine learning model and based at least in part on the historical member vertices, historical member query vertices from the healthcare graph data object, generating, based at least in part on the historical member query vertices, predicted member query vertices in the healthcare graph data object, and performing one or more prediction-based actions based at least in part on the one or more predicted member query vertices.

### TECHNOLOGICAL FIELD

Embodiments of the present disclosure relate generally to improving functionalities of systems and devices such as, but not limited to, graph-based data systems. For example, various embodiments of the present disclosure may programmatically generate predicted member query vertices for a healthcare graph data object and perform one or more prediction-based actions based at least in part on the predicted member query vertices.

### BACKGROUND

There are many technical problems and challenges associated with databases and data systems that are configured to, for example, store and/or manage data and/or information related to healthcare. For example, many databases and data systems do not have the capability to generate predicted data that is personalized based on information associated with a member who uses the system.

### BRIEF SUMMARY

In general, embodiments of the present disclosure provide methods, apparatuses, systems, computing devices, computing entities, and/or the like.

In accordance with various embodiments of the present disclosure, an apparatus is provided. The apparatus may comprise at least one processor and at least one non-transitory memory comprising a computer program code. The at least one non-transitory memory and the computer program code may be configured to, with the at least one processor, cause the apparatus to generate one or more edges connecting one or more attribute vertices to a member vertex in a healthcare graph data object based at least in part on one or more member data objects comprising at least one of a member demographics data object, a member history data object, or a member symptom data object; determine, using at least one graph-based machine learning model and based at least in part on the one or more attribute vertices, one or more historical member vertices from the healthcare graph data object at least according to one or more resemblance metrics associated with the one or more historical member vertices and relative to the member vertex; determine, using the at least one graph-based machine learning model and based at least in part on the one or more historical member vertices, one or more historical member query vertices from the healthcare graph data object at least according to one or more prioritization metrics associated with the one or more historical member query vertices; generate, based at least in part on the one or more historical member query vertices, one or more predicted member query vertices in the healthcare graph data object; and perform one or more prediction-based actions based at least in part on the one or more predicted member query vertices.

In some embodiments, the member vertex is associated with a member identifier. In some embodiments, the at least one non-transitory memory and the computer program code are configured to, with the at least one processor, cause the apparatus to: retrieve at least one electronic health record data object associated with the member identifier, wherein the at least one electronic health record data object comprises demographic information and health history information associated with the member identifier; generate the member demographics data object based at least in part on the demographic information from the at least one electronic health record data object; and generate the member history data object based at least in part on the health history information from the at least one electronic health record data object.

In some embodiments, the member vertex is associated with a member identifier. In some embodiments, the at least one non-transitory memory and the computer program code are configured to, with the at least one processor, cause the apparatus to: prior to generating the one or more attribute vertices, receive an actual member visit indication from a client computing entity associated with the member identifier; and in response to receiving the actual member visit indication, generate a visit vertex in the healthcare graph data object that is connected to the member vertex.

In some embodiments, the at least one non-transitory memory and the computer program code are configured to, with the at least one processor, cause the apparatus to: receive, from the client computing entity associated with the member identifier, one or more audio inputs or one or more visual inputs; generate one or more textual inputs based at least in part on the one or more audio inputs or the one or more visual inputs; extract, using at least one natural language processing model, one or more supplemental data inputs from the one or more textual inputs; and generate one or more member supplemental data objects based at least in part on the one or more supplemental data inputs.

In some embodiments, the one or more member data objects comprise the one or more member supplemental data objects. In some embodiments, the at least one non-transitory memory and the computer program code are configured to, with the at least one processor, cause the apparatus to: generate the one or more attribute vertices based on the one or more member supplemental data objects.

In some embodiments, the at least one non-transitory memory and the computer program code are configured to, with the at least one processor, cause the apparatus to: calculate the one or more resemblance metrics based at least in part on the one or more attribute vertices associated with the member vertex and an attribute vertex set associated with the one or more historical member vertices.

In some embodiments, the healthcare graph data object comprises a member subgraph and one or more historical member subgraphs. In some embodiments, the member subgraph comprises the member vertex and the one or more attribute vertices that are connected to the member vertex and associated with the member vertex. In some embodiments, each of the one or more historical member subgraphs comprises a historical member vertex of the one or more historical member vertices and at least one attribute vertex from the attribute vertex set that is connected to the historical member vertex and is associated with the historical member vertex.

In some embodiments, when calculating the one or more resemblance metrics, the at least one non-transitory memory and the computer program code are configured to, with the at least one processor, cause the apparatus to: calculate one or more cosine similarity measures between the member subgraph of the healthcare graph data object and each of the one or more historical member subgraphs of the healthcare graph data object; and determine the one or more resemblance metrics based at least in part on the one or more cosine similarity measures.

In some embodiments, when calculating the one or more resemblance metrics, the at least one non-transitory memory and the computer program code are configured to, with the at least one processor, cause the apparatus to: train the at least one graph-based machine learning model based at least in part on one or more training healthcare graph data objects and one or more training resemblance metrics; subsequent to training the at least one graph-based machine learning model, input the healthcare graph data object to the at least one graph-based machine learning model; and receive, from the at least one graph-based machine learning model, the one or more resemblance metrics. In some embodiments, each of the one or more training healthcare graph data objects comprises a plurality of training member subgraphs. In some embodiments, each of the one or more resemblance metrics indicates a similarity measure between one of the one or more historical member vertices and the member vertex.

In some embodiments, a historical member query vertex of the one or more historical member query vertices is connected to a historical member vertex of the one or more historical member vertices in the healthcare graph data object via one or more edges. In some embodiments, the at least one non-transitory memory and the computer program code are configured to, with the at least one processor, cause the apparatus to: determine, for the historical member query vertex, a member-query edge weight associated with the one or more edges; and determine, for the historical member query vertex, a prioritization metrics of the one or more prioritization metrics based at least in part on the member-query edge weight.

In some embodiments, when determining the member-query edge weight, the at least one non-transitory memory and the computer program code are configured to, with the at least one processor, cause the apparatus to: determine a healthcare outcome measure associated with the historical member vertex; and calculate the member-query edge weight based at least in part on the healthcare outcome measure.

In some embodiments, when determining the member-query edge weight, the at least one non-transitory memory and the computer program code are configured to, with the at least one processor, cause the apparatus to: determine a healthcare cost measure associated with the historical member vertex; and calculate the member-query edge weight based at least in part on the healthcare cost measure.

In some embodiments, the healthcare graph data object comprises a plurality of historical visit vertices. In some embodiments, a historical visit vertex of the plurality of historical visit vertices is connected to the historical member vertex and the historical member query vertex.

In some embodiments, the one or more edges comprise a member-visit edge connecting the historical member vertex to the historical visit vertex and a visit-query edge connecting the historical visit vertex to the historical member query vertex.

In some embodiments, the member-query edge weight is a combination of a member-visit edge weight associated with the member-visit edge and a visit-query edge weight associated with the visit-query edge.

In some embodiments, when performing the one or more prediction-based actions based at least in part on the one or more predicted member query vertices, the at least one non-transitory memory and the computer program code are configured to, with the at least one processor, cause the apparatus to: render, on a graphic user interface displayed on a client computing entity, one or more predicted member query indications based at least in part on the one or more predicted member query vertices; and subsequent to rendering the one or more predicted member query indications, receive a user input associated with a predicted member query indication of the one or more predicted member query indications that corresponds to a predicted member query vertex of the one or more predicted member query vertices.

In some embodiments, the at least one non-transitory memory and the computer program code are configured to, with the at least one processor, cause the apparatus to: determine that the user input indicates a user interest of the predicted member query indication; determine a historical member query vertex corresponds to the predicted member query vertex; and increase a member-query edge weight of a member-query edge associated with the historical member query vertex.

In some embodiments, the at least one non-transitory memory and the computer program code are configured to, with the at least one processor, cause the apparatus to: determine that the user input indicates a user disinterest of the predicted member query indication; determine a historical member query vertex corresponds to the predicted member query vertex; and decrease a member-query edge weight of a member-query edge associated with the historical member query vertex.

In accordance with various embodiments of the present disclosure, a computer-implemented method is provided. The computer-implemented method may comprise generating one or more edges connecting one or more attribute vertices to a member vertex in a healthcare graph data object based at least in part on one or more member data objects comprising at least one of a member demographics data object, a member history data object, or a member symptom data object; determining, using at least one graph-based machine learning model and based at least in part on the one or more attribute vertices, one or more historical member vertices from the healthcare graph data object at least according to one or more resemblance metrics associated with the one or more historical member vertices and relative to the member vertex; determining, using the at least one graph-based machine learning model and based at least in part on the one or more historical member vertices, one or more historical member query vertices from the healthcare graph data object at least according to one or more prioritization metrics associated with the one or more historical member query vertices; generating, based at least in part on the one or more historical member query vertices, one or more predicted member query vertices in the healthcare graph data object; and performing one or more prediction-based actions based at least in part on the one or more predicted member query vertices.

In accordance with various embodiments of the present disclosure, a computer program product is provided. The computer program product may comprise at least one non-transitory computer-readable storage medium having computer-readable program code portions stored therein. The computer-readable program code portions may comprise an executable portion configured to generate one or more edges connecting one or more attribute vertices to a member vertex in a healthcare graph data object based at least in part on one or more member data objects comprising at least one of a member demographics data object, a member history data object, or a member symptom data object; determine, using at least one graph-based machine learning model and based at least in part on the one or more attribute vertices, one or more historical member vertices from the healthcare graph data object at least according to one or more resemblance metrics associated with the one or more historical member vertices and relative to the member vertex; determine, using the at least one graph-based machine learning model and based at least in part on the one or more historical member vertices, one or more historical member query vertices from the healthcare graph data object at least according to one or more prioritization metrics associated with the one or more historical member query vertices; generate, based at least in part on the one or more historical member query vertices, one or more predicted member query vertices in the healthcare graph data object; and perform one or more prediction-based actions based at least in part on the one or more predicted member query vertices.

The above summary is provided merely for purposes of summarizing some example embodiments to provide a basic understanding of some aspects of the disclosure. Accordingly, it will be appreciated that the above-described embodiments are merely examples. It will be appreciated that the scope of the disclosure encompasses many potential embodiments in addition to those here summarized, some of which will be further described below.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 is a diagram of an example graph-based query prediction platform/system that can be used in accordance with various embodiments of the present disclosure;

FIG. 2 is a schematic representation of an example query prediction computing entity in accordance with various embodiments of the present disclosure;

FIG. 3 is a schematic representation of an example client computing entity in accordance with various embodiments of the present disclosure;

FIG. 4, FIG. 5, FIG. 6A, FIG. 6B, FIG. 7, FIG. 8, FIG. 9, FIG. 10, FIG. 11, FIG. 12, FIG. 13, FIG. 14A, and FIG. 14B provide example flowcharts and diagrams illustrating example steps, processes, procedures, and/or operations associated with an example graph-based query prediction platform/system in accordance with various embodiments of the present disclosure; and

FIG. 15, FIG. 16, FIG. 17, FIG. 18, and FIG. 19 provide example views of example graphic user interfaces in accordance with various embodiments of the present disclosure.

### DETAILED DESCRIPTION OF SOME EXAMPLE EMBODIMENTS

Various embodiments of the present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers may refer to like elements throughout. The phrases “in one embodiment,” “according to one embodiment,” and/or the like generally mean that the particular feature, structure, or characteristic following the phrase may be included in at least one embodiment of the present disclosure and may be included in more than one embodiment of the present disclosure (importantly, such phrases do not necessarily may refer to the same embodiment).

### I. COMPUTER PROGRAM PRODUCTS, METHODS, AND COMPUTING ENTITIES

Embodiments of the present disclosure may be implemented as computer program products that comprise articles of manufacture. Such computer program products may include one or more software components including, for example, applications, software objects, methods, data structures, and/or the like. A software component may be coded in any of a variety of programming languages. An illustrative programming language may be a lower-level programming language such as an assembly language associated with a particular hardware architecture and/or operating system platform/system. A software component comprising assembly language instructions may require conversion into executable machine code by an assembler prior to execution by the hardware architecture and/or platform/system. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Additionally, or alternatively, embodiments of the present disclosure may be implemented as a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media may include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### II. EXEMPLARY SYSTEM ARCHITECTURE

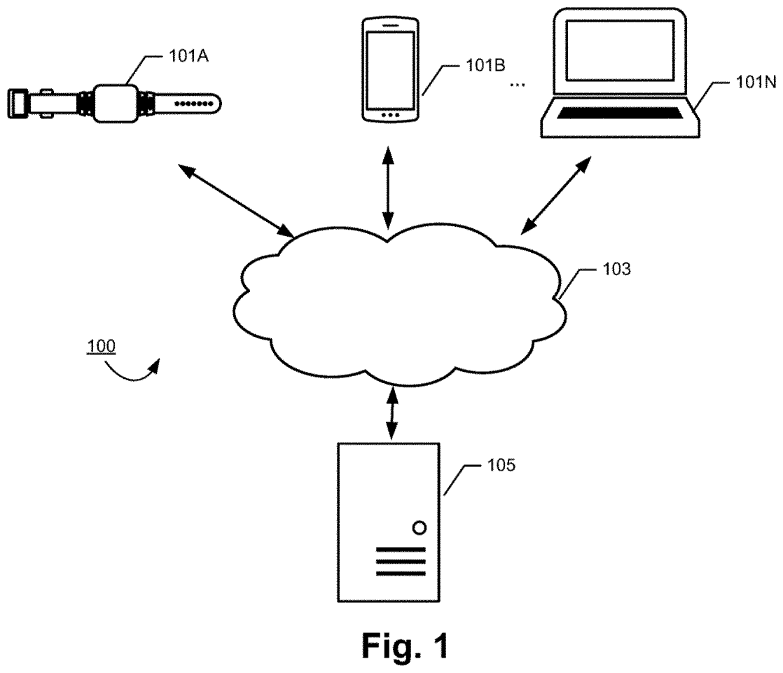

FIG. 1 provides an illustration of a graph-based query prediction platform/system 100 that can be used in conjunction with various embodiments of the present disclosure. As shown in FIG. 1, the graph-based query prediction platform/system 100 may comprise apparatuses, devices, and components such as, but not limited to, one or more client computing entities 101A, 101B, . . . 101N, one or more query prediction computing entities 105 and one or more networks 103.

Each of the components of the graph-based query prediction platform/system 100 may be in electronic communication with, for example, one another over the same or different wireless or wired networks 103 including, for example, a wired or wireless Personal Area Network (PAN), Local Area Network (LAN), Metropolitan Area Network (MAN), Wide Area Network (WAN), and/or the like. For example, the one or more client computing entities 101A . . . 101N and the one or more query prediction computing entities 105 may be in electronic communication with one another to exchange data and information. Additionally, while FIG. 1 illustrates certain system entities as separate, standalone entities, the various embodiments are not limited to this particular architecture.

### a. Exemplary Query Prediction Computing Entity

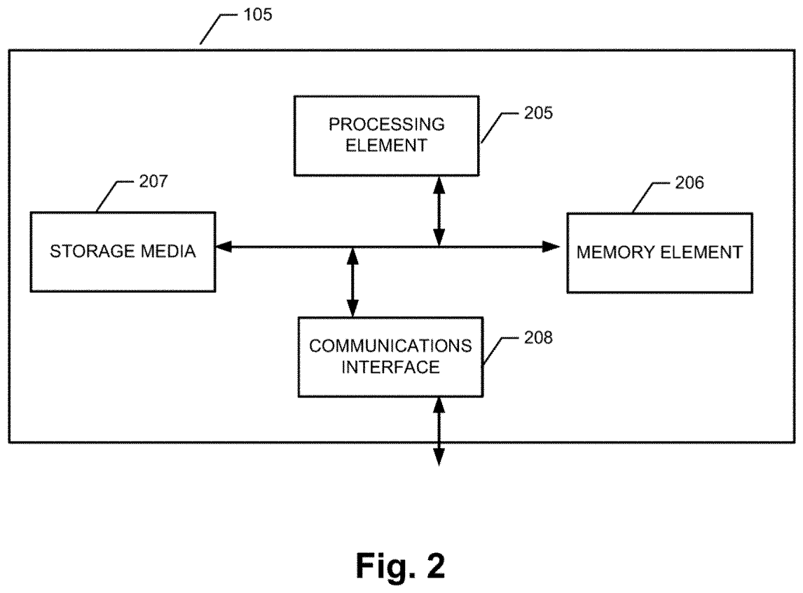

FIG. 2 provides a schematic of a query prediction computing entity 105 according to one embodiment of the present disclosure. In general, the terms computing entity, entity, device, system, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein. In one embodiment, these functions, operations, and/or processes can be performed on data, content, information, and/or similar terms used herein.

As indicated, in one embodiment, the query prediction computing entity 105 may also include one or more network and/or communications interface 208 for communicating with various computing entities, such as by communicating data, content, information, and/or similar terms used herein that can be transmitted, received, operated on, processed, displayed, stored, and/or the like. For instance, the query prediction computing entity 105 may communicate with other query prediction computing entities 105 and one or more client computing entities 101A-101N and/or the like.

As shown in FIG. 2, in one embodiment, the query prediction computing entity 105 may include or be in communication with one or more processing elements (for example, processing element 205) (also referred to as processors, processing circuitry, and/or similar terms used herein interchangeably) that communicate with other elements within the query prediction computing entity 105 via a bus, for example, or network connection. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

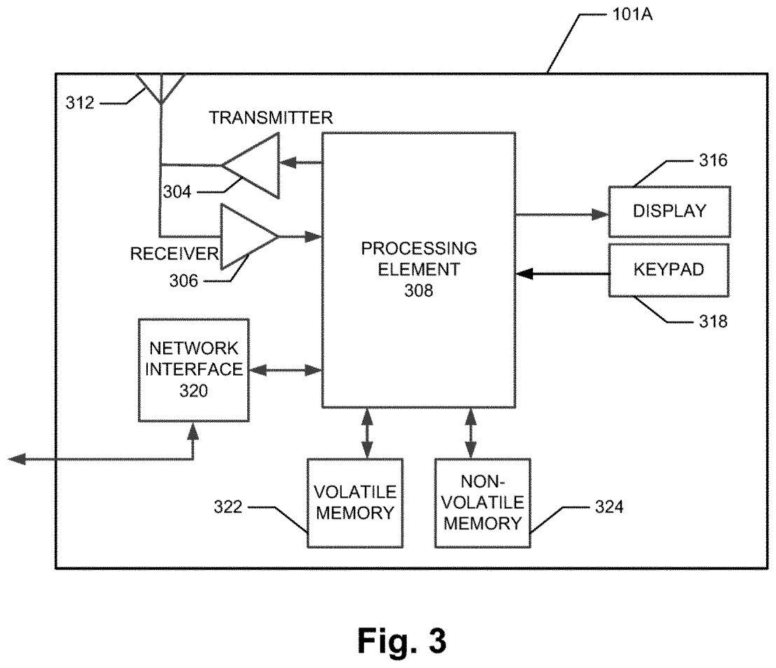

In one embodiment, the query prediction computing entity 105 may further include or be in communication with volatile media (also referred to as volatile storage, memory, memory storage, memory circuitry and/or similar terms used herein interchangeably). In one embodiment, the volatile storage or memory may also include one or more memory element 206 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory element 206 may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205 as shown in FIG. 2 and/or the processing element 308 as described in connection with FIG. 3. Thus, the databases, database instances, database management system entities, data, applications, programs, program modules, scripts, source code, object code, byte code, compiled code, interpreted code, machine code, executable instructions, and/or the like may be used to control certain aspects of the operation of the query prediction computing entity 105 with the assistance of the processing element 205 and operating system.

In one embodiment, the query prediction computing entity 105 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or storage media 207 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or storage media 207 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to may refer to a structured or unstructured collection of information/data that is stored in a computer-readable storage medium.

Storage media 207 may also be embodied as a data storage device or devices, as a separate database server or servers, or as a combination of data storage devices and separate database servers. Further, in some embodiments, storage media 207 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. An example of the embodiments contemplated herein would include a cloud data storage system maintained by a third-party provider and where some or all of the information/data required for the operation of the recovery system may be stored. Further, the information/data required for the operation of the recovery system may also be partially stored in the cloud data storage system and partially stored in a locally maintained data storage system. More specifically, storage media 207 may encompass one or more data stores configured to store information/data usable in certain embodiments.

As indicated, in one embodiment, the query prediction computing entity 105 may also include one or more network and/or communications interface 208 for communicating with various computing entities, such as by communicating data, content, information, and/or similar terms used herein interchangeably that can be transmitted, received, operated on, processed, displayed, stored, and/or the like. For instance, the query prediction computing entity 105 may communicate with computing entities or communication interfaces of other query prediction computing entities 105, client computing entities 101A-101N, and/or the like.

As indicated, in one embodiment, the query prediction computing entity 105 may also include one or more network and/or communications interface 208 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the query prediction computing entity 105 may be configured to communicate via wireless external communication networks using any of a variety of protocols, such as general packet radio service (GPRS), Universal Mobile Telecommunications System (UMTS), Code Division Multiple Access 1900 (CDMA1900), CDMA1900 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), Institute of Electrical and Electronics Engineers (IEEE) 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The query prediction computing entity 105 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the query prediction computing entity's components may be located remotely from components of other query prediction computing entities 105, such as in a distributed system. Furthermore, one or more of the components may be aggregated and additional components performing functions described herein may be included in the query prediction computing entity 105. Thus, the query prediction computing entity 105 can be adapted to accommodate a variety of needs and circumstances.

### b. Exemplary Client Computing Entity

FIG. 3 provides an illustrative schematic representative of one of the client computing entities 101A to 101N that can be used in conjunction with embodiments of the present disclosure. As will be recognized, the client computing entity may be operated by an agent and include components and features similar to those described in conjunction with the query prediction computing entity 105. Further, as shown in FIG. 3, the client computing entity may include additional components and features. For example, the client computing entity 101A can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various entities, such as a query prediction computing entity 105, another client computing entity 101A, and/or the like. In this regard, the client computing entity 101A may be capable of operating with one or more air interface standards, communication protocols, modulation types, and access types. More particularly, the client computing entity 101A may comprise a network interface 320, and may operate in accordance with any of a number of wireless communication standards and protocols. In a particular embodiment, the client computing entity 101A may operate in accordance with multiple wireless communication standards and protocols, such as GPRS, UMTS, CDMA1900, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the client computing entity 101A can communicate with various other entities using Unstructured Supplementary Service data (USSD), Short Message Service (SMS), Multimedia Messaging Service (MMS), Dual-Tone Multi-Frequency (DTMF) Signaling, Subscriber Identity Module Dialer (SIM dialer), and/or the like. The client computing entity 101A can also download changes, add-ons, and updates, for instance, to its firmware, software (e.g., including executable instructions, applications, program modules), and operating system.

According to one embodiment, the client computing entity 101A may include location determining aspects, devices, modules, functionalities, and/or similar words used herein interchangeably. For example, the client computing entity 101A may include outdoor positioning aspects, such as a location module adapted to acquire, for example, latitude, longitude, altitude, geocode, course, direction, heading, speed, UTC, date, and/or various other information/data. In one embodiment, the location module can acquire data, sometimes known as ephemeris data, by identifying the number of satellites in view and the relative positions of those satellites. The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. Alternatively, the location information/data/data may be determined by triangulating the position in connection with a variety of other systems, including cellular towers, Wi-Fi access points, and/or the like. Similarly, the client computing entity 101A may include indoor positioning aspects, such as a location module adapted to acquire, for example, latitude, longitude, altitude, geocode, course, direction, heading, speed, time, date, and/or various other information/data. Some of the indoor aspects may use various position or location technologies including Radio-Frequency Identification (RFID) tags, indoor beacons or transmitters, Wi-Fi access points, cellular towers, nearby computing devices (e.g., smartphones, laptops) and/or the like. For instance, such technologies may include iBeacons, Gimbal proximity beacons, Bluetooth Low Energy (BLE) transmitters, Near Field Communication (NFC) transmitters, and/or the like. These indoor positioning aspects can be used in a variety of settings to determine the location of someone or something to within inches or centimeters.

The client computing entity 101A may also comprise a user interface comprising one or more user input/output interfaces (e.g., a display 316 and/or speaker/speaker driver coupled to a processing element 308 and a touch screen, keyboard, mouse, and/or microphone coupled to a processing element 308). For example, the user output interface may be configured to provide an application, browser, user interface, dashboard, webpage, and/or similar words used herein interchangeably executing on and/or accessible via the client computing entity 101A to cause display or audible presentation of information/data and for user interaction therewith via one or more user input interfaces. The user output interface may be updated dynamically from communication with the query prediction computing entity 105. The user input interface can comprise any of a number of devices allowing the client computing entity 101A to receive data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the client computing entity 101A and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the client computing entity 101A can collect information/data, user interaction/input, and/or the like.

The client computing entity 101A can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the client computing entities 101A-101N.

### c. Exemplary Networks

In one embodiment, the networks 103 may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks 103 may have any suitable communication range associated therewith and may include, for example, global networks (e.g., the Internet), MANs, WANs, LANs, or PANs. In addition, the networks 103 may include medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms/systems provided by network providers or other entities.

Further, the networks 103 may utilize a variety of networking protocols including, but not limited to, TCP/IP based networking protocols. In some embodiments, the protocol is a custom protocol of JavaScript Object Notation (JSON) objects sent via a WebSocket channel. In some embodiments, the protocol is JSON over RPC, JSON over REST/HTTP, and/or the like.

### III. EXEMPLARY OPERATION

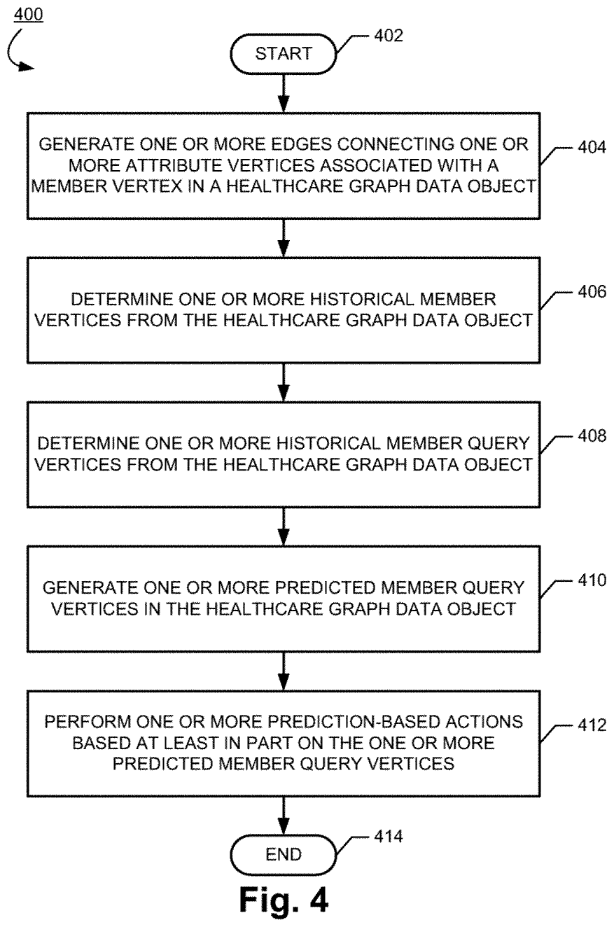

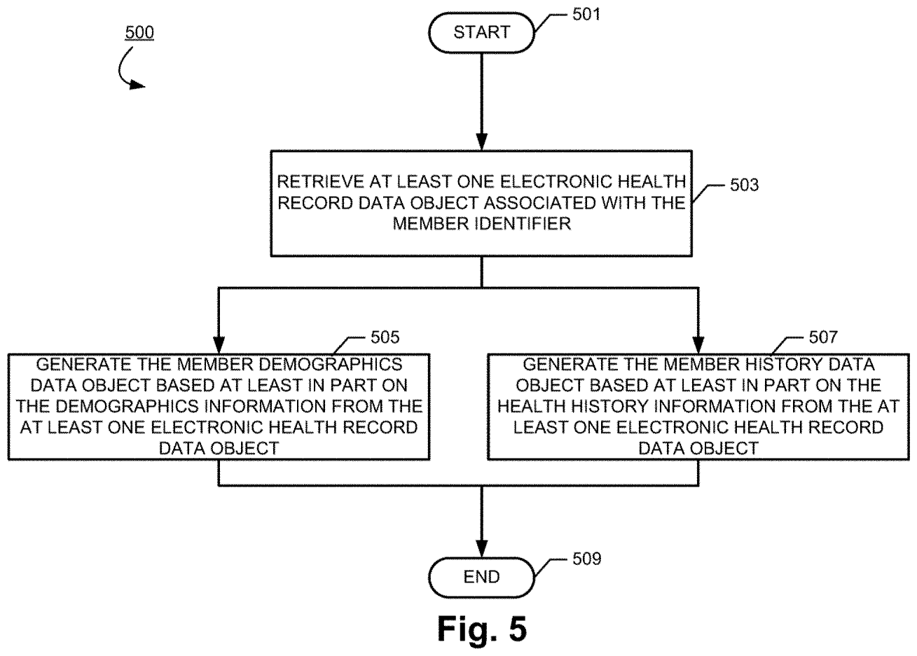

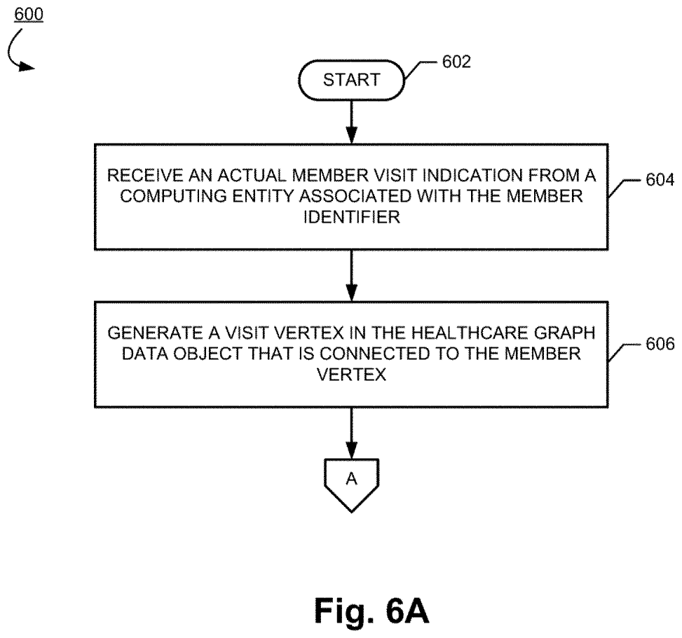

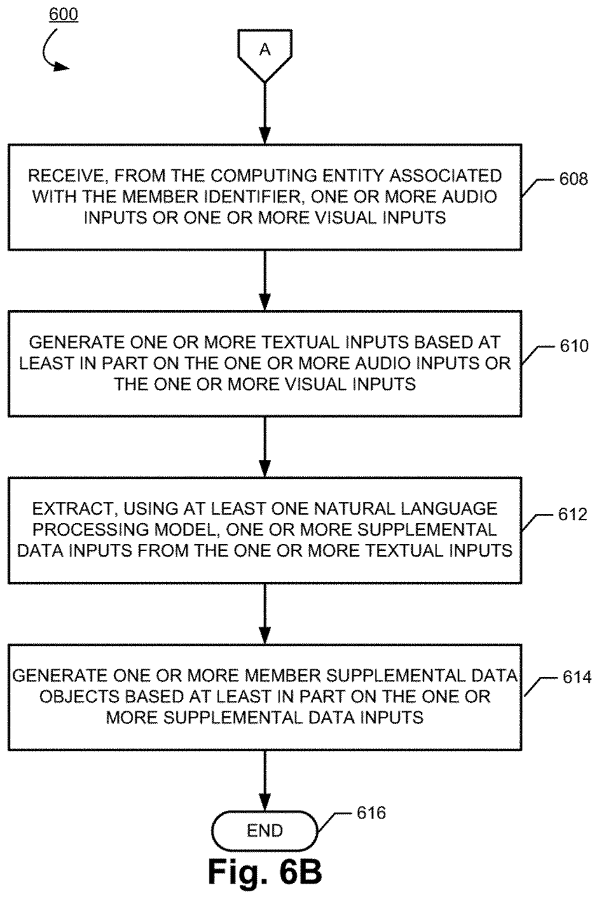

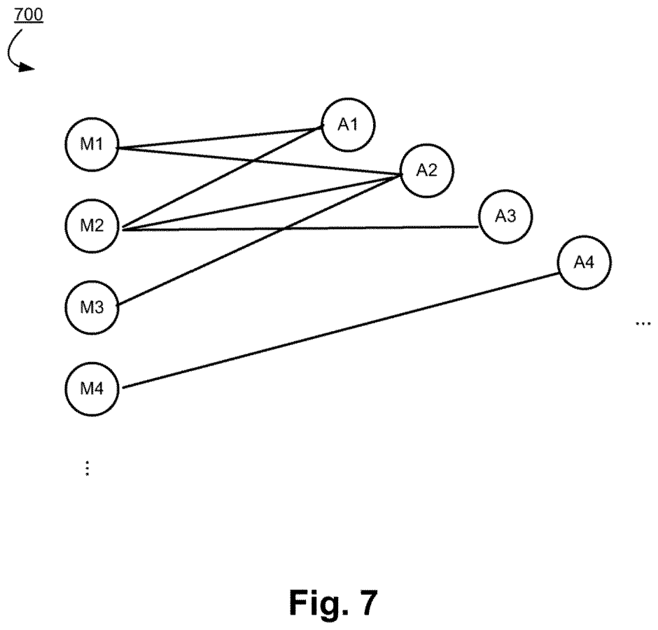

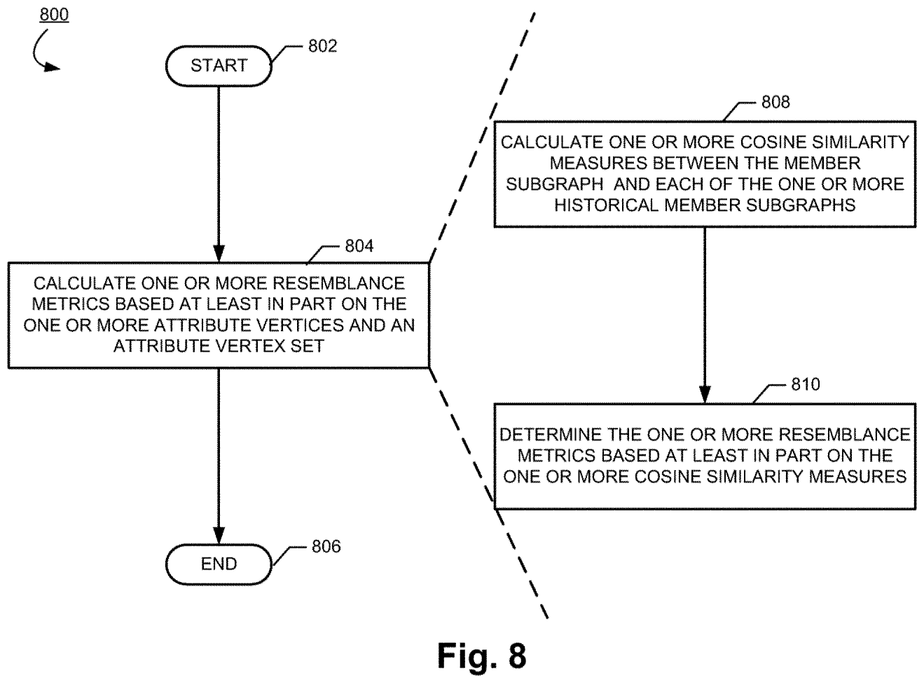

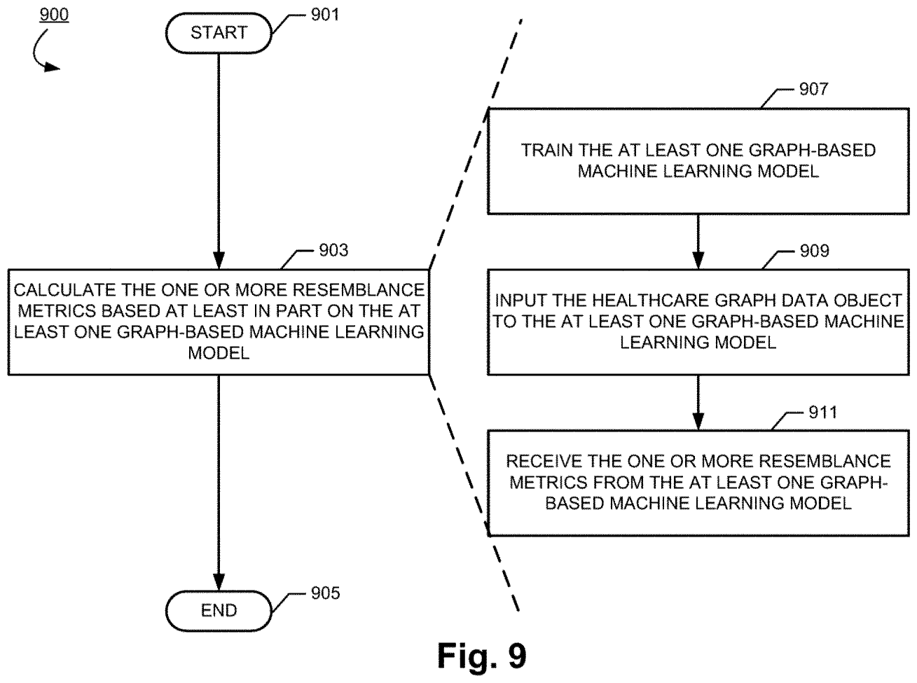

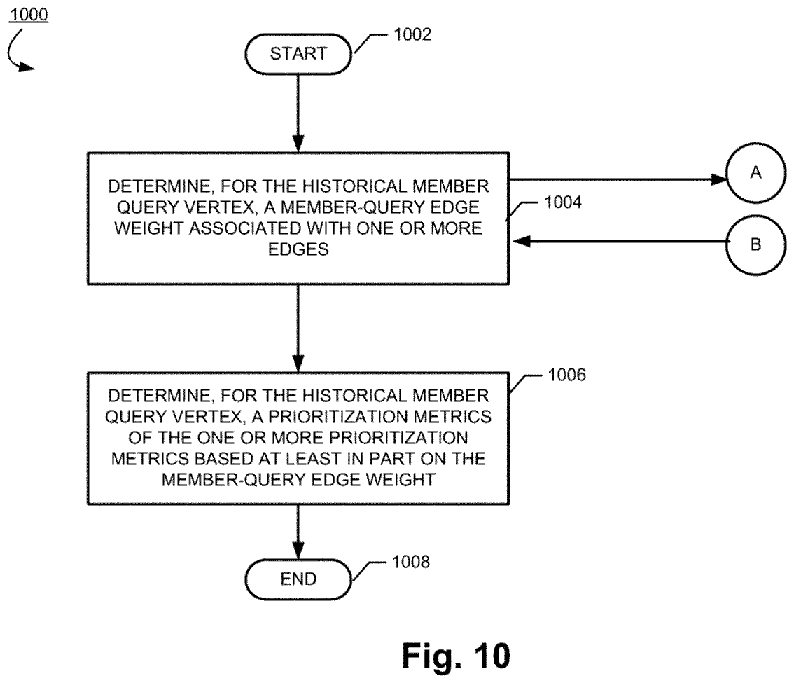

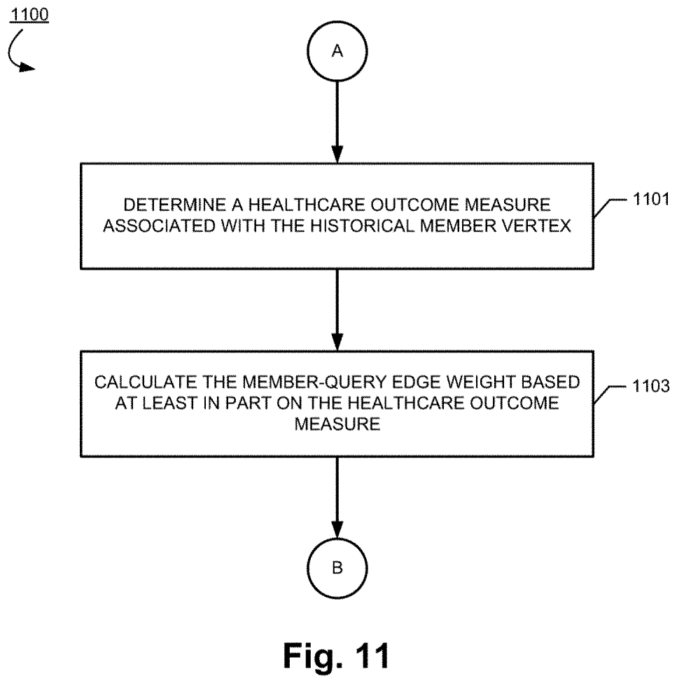

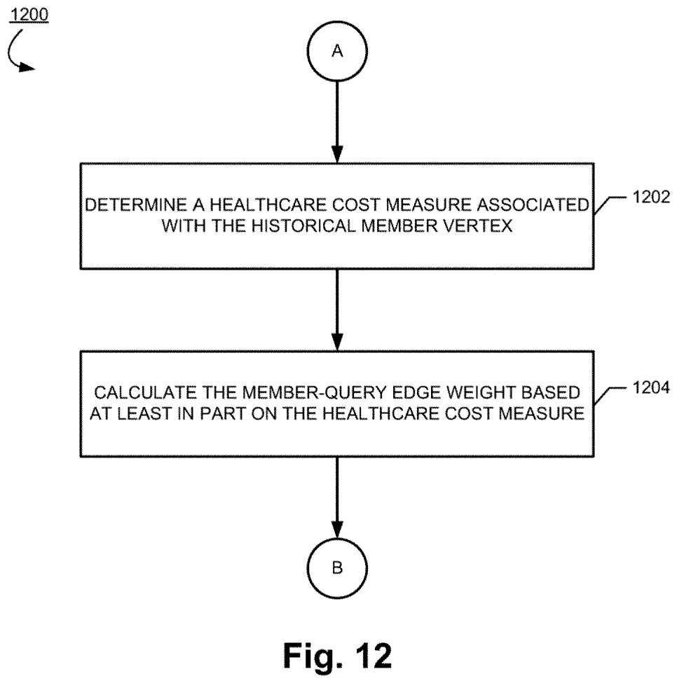

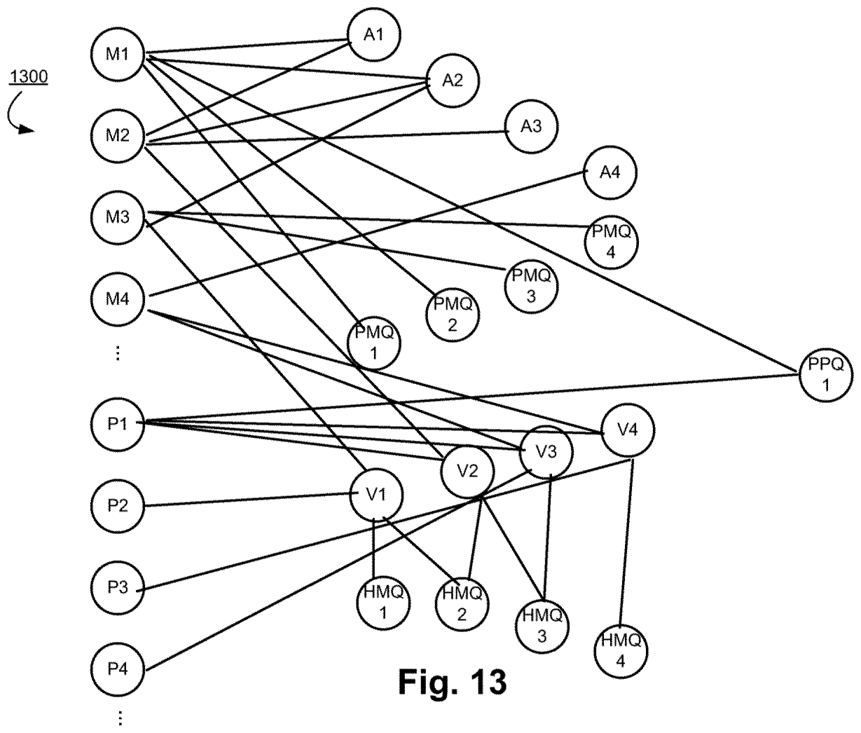

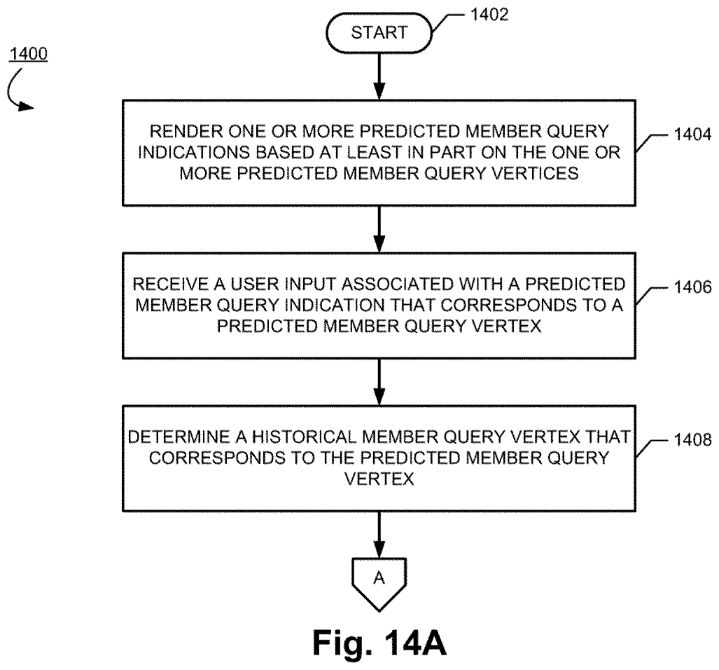

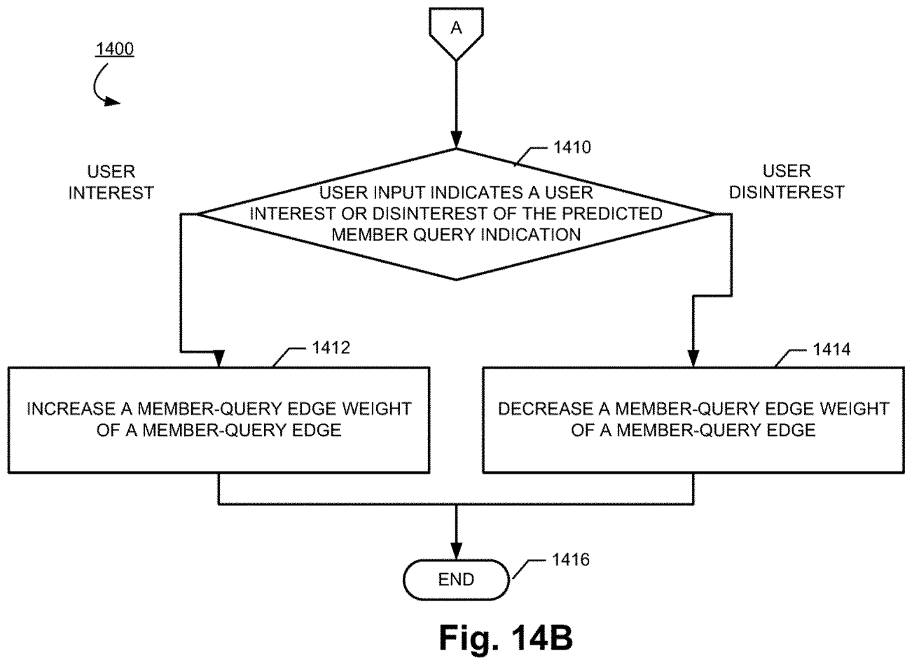

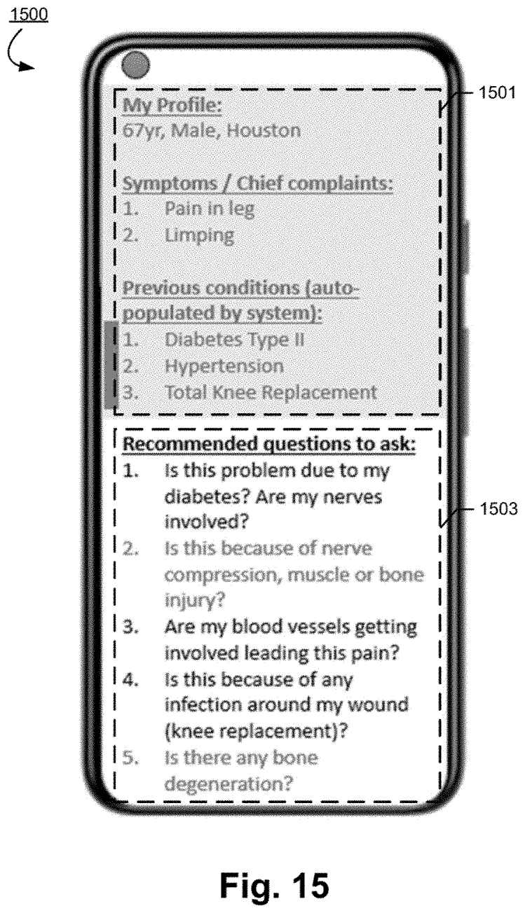

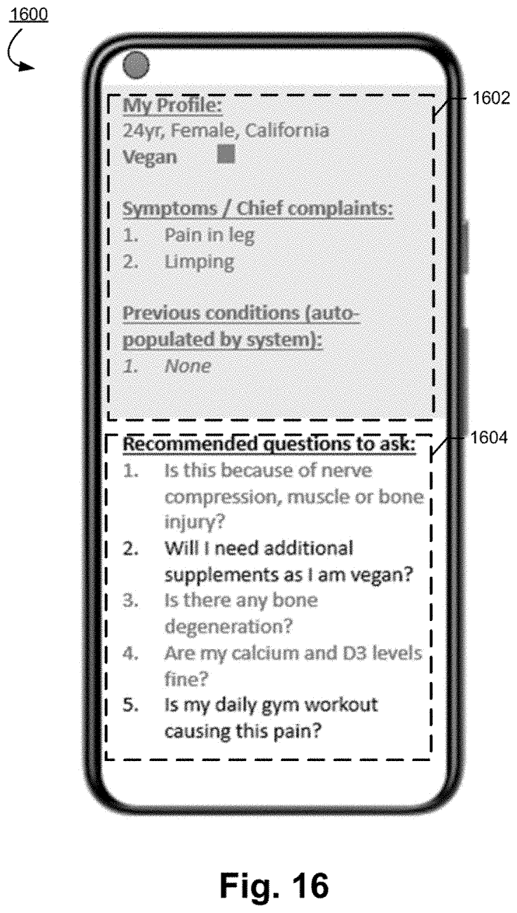

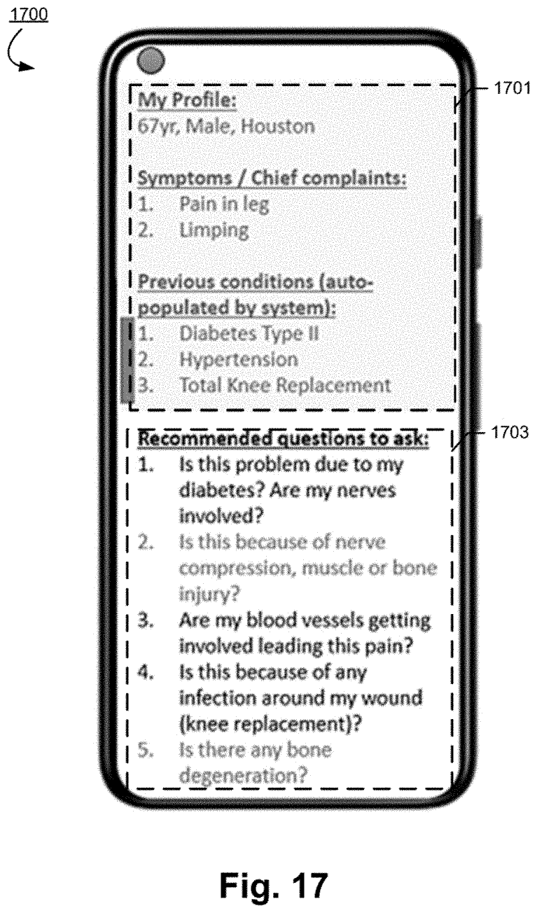

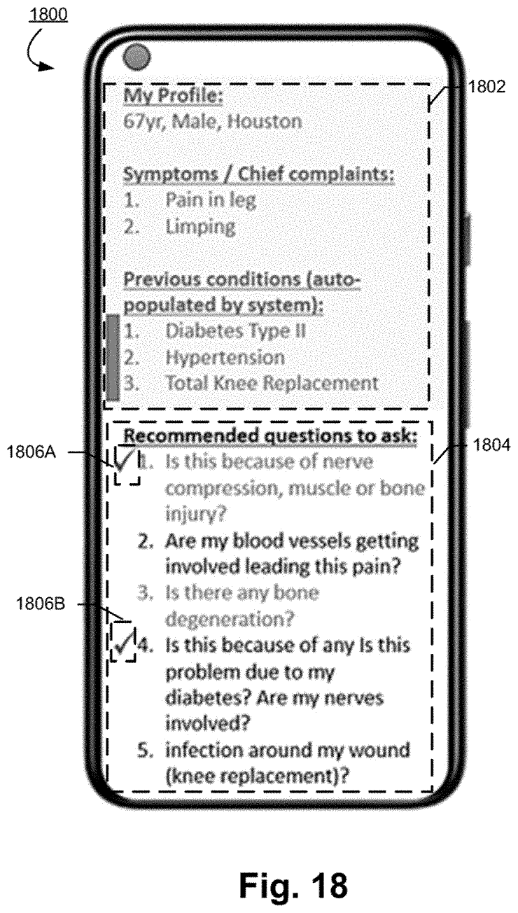

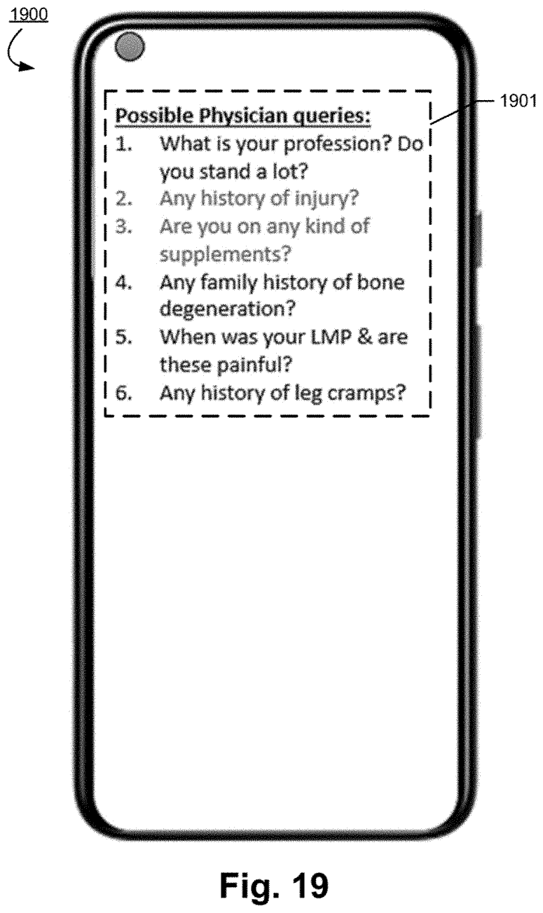

Reference will now be made to FIG. 4, FIG. 5, FIG. 6A, FIG. 6B, FIG. 7, FIG. 8, FIG. 9, FIG. 10, FIG. 11, FIG. 12, FIG. 13, FIG. 14A, and FIG. 14B, which provide flowcharts and diagrams illustrating example steps, processes, procedures, and/or operations associated with an example graph-based query prediction platform/system and/or an example query prediction computing entity in accordance with various embodiments of the present disclosure. FIG. 15, FIG. 16, FIG. 17, FIG. 18, and FIG. 19 provide example views of interactive user interfaces in accordance with various embodiments of the present disclosure.

While example embodiments of the present disclosure may be described in the context of healthcare, a person of ordinary skill in the relevant technology will recognize that embodiments of the present disclosure are not limited to this context only.

### a. Overview and Technical Advantages

A graph data system refers to a data system that uses graph structures with nodes, edges, and properties to represent, store, and analyze data and/or information. Graph data systems have great potential in the field of machine learning and predictive analysis.

For example, patients (also referred to as members) often do not take full advantage of the opportunity to ask questions during office visits with their provider (e.g. doctor, pharmacist, etc.). In many cases, members may simply not understand what questions would be valuable. In other cases, members may be intimidated by the process or simply forget to ask relevant questions that they remember at some point after the office visit has concluded. In many situations, it is often difficult to reconnect with a provider to ask those clarifying questions. In such an example, a graph data system may be implemented to generate recommended questions for a member to ask a provider during the member's visit to the provider, and/or for the provider to ask the member during the member's visit to the provider.

However, many graph data systems are plagued by technical challenges and difficulties.

For example, many machine learning and predictive analysis implementations require generating predicted data that are customized based on the individualized data and/or information associated with a particular user. As an example, an example data system may comprise data and/or information associated with a user Alex and another user Brandon. While some data and/or information may be associated with both Alex and Brandon, they may each be associated with certain different data and/or information. Many systems may generate predicted data that have lower relevancy, applicability, and accuracy for a particular user.

Continuing from the healthcare example above, some data systems may generate lists of suggested questions for a member to ask a provider based on the symptoms that the member may have. While some of these questions may be helpful, they may have variable relevancy, applicability, and accuracy to the member.

In contrast, various embodiments of the present disclosure overcome these technical challenges and difficulties, and may provide various technical advantages. For example, various embodiments of the present disclosure may increase the relevancy, applicability, and accuracy of predicted data compared to those of other data systems by using at least one graph-based machine learning model and based at least in part on determining resemblance metrics and prioritization metrics.

In some embodiments, various example embodiments of the present disclosure may update a healthcare graph data object (that includes member vertices and attribute vertices) and train a graph-based machine learning model based on the healthcare graph data object to dynamically generate predicted member query vertices and/or predicted provider query vertices. For example, various embodiments of the present disclosure describe a system for recommending specific questions for individual members to ask providers, and in some cases for recommending specific questions for providers to ask members.

In some embodiments, an example graph-based query prediction platform/system in accordance with examples of the present disclosure may analyze data from past members in similar medical, environmental and demographic circumstances, and present the member with specific questions he or she may ask during a particular visit. In some embodiments, this list of questions can be dynamically updated as the member and the provider progress in their conversation. In some cases, the example graph-based query prediction platform/system can reveal to the member anticipatory questions that the provider may ask, and in other cases it can reveal questions to the provider that are of specific relevance to the member.

In some embodiments, the graph-based query prediction platform/system may take, as an input, all information on the member's medical conditions, prescriptions, and demographic information. In some embodiments, this information is arranged in a network or graph structure (such as a graph with healthcare information).

In some embodiments, at the onset of the visit, the member launches an application on their computing entity that is connected to the graph-based query prediction platform/system. The platform/system monitors the conversation between the member with their provider. The platform/system optionally analyzes photographs of any printed materials given to the member before, during, or after the office visit.

In some embodiments, the content of the monitored conversation and the scanned materials are processed (e.g. speech-to-text, optical character recognition (OCR)), and the text of these inputs are processed by natural language processing (NLP) techniques (such as RAKE) to extract keywords and identify the most important parts of the member/provider interaction.

In some embodiments, this information is added to a graph data object in the form of, such as, but not limited to, a labelled property graph that contains healthcare information. In some embodiments, the graph-based query prediction platform/system adds new vertices for each visit. In some embodiments, each visit vertex and/or each member vertex may have associated attribute vertices described above. In some embodiments, these attribute vertices complement other attribute vertices that are generated based on member information such as disease diagnosis, demographic information, and so forth. While the description above provides an example of a labelled property graph, it is noted that the scope of the present disclosure is not limited to the example above. In some embodiments, an example graph data object may be in other forms and/or types (for example, any graph database types including, but not limited to, knowledge graphs such as, but not limited to, Semantic Knowledge Graphs).

In some embodiments, the graph-based query prediction platform/system may then perform a graph similarity calculation to determine how much the current member relates to other members (for example, based on similarity measures such as, but not limited to, cosine similarity measures, Jaccard similarity measures, etc., and/or based on graph-neural networks or other model-based similarity determination mechanisms).

In some embodiments, the graph-based query prediction platform/system recommends questions that have been asked by other members to which they are most similar. Part of this calculation may factor in the outcome of the treatment path so that questions from those members with positive outcomes or improvements are weighted above those with negative outcomes. In the absence of a specific code, lack of follow-up visits can serve as a surrogate for positive outcome.

In some embodiments, the questions are then ranked by the strength of similarity (e.g. based on resemblance metrics and/or prioritization metrics described herein). For example, in some embodiments, the topmost match may have the most highly ranked questions, the second most match will have questions ordered next, and so forth.

In some embodiments, the recommended questions are displayed on a computing entity (such as a member's device), and members may choose to ask the question or skip the question. In some embodiments, either of these actions will cause the list of questions to refresh with the next set of questions in the list. In some embodiments, lower ranked questions may move up the list. In some embodiments, questions already asked will be indicated by a check mark or removed from the list.

In some embodiments, the member's question selection can be used to weight the edges between their member vertex and the query vertex in the graph. This may have the effect of strengthening future recommendations for other members.

In some embodiments, the system may generate a list of questions that the provider may ask the member, and those questions can be presented to the member prior to the visit in order to prepare the member to answer these provider questions. For example, the system may inform the member that the provider is likely to ask, “did you remember to fast?” in order to prompt the user to take action to make the visit more effective.

As an example, a member may arrange for an office visit because they suspect the onset of an infection. In some embodiments, the graph-based query prediction platform/system provides the member with a pre-visit prompt such as to log fevers every few hours before the visit in order to satisfy the anticipated provider question.

As another example, the member is scheduled to check into the hospital for a procedure. In some embodiments, the graph-based query prediction platform/system provides the member a pre-visit prompt to fast beforehand as this is required for the procedure. The provider may ask if this has been done, and not doing so would add delays to the procedure being performed. In this way, the graph-based query prediction platform/system provides a savings benefit to the enterprise.

In some embodiments, the system may periodically assess the health of members who had used the system, and the questions they asked. In some embodiments, members whose care cost more and/or whose recovery times were longer would have their question edges weight adjusted downward. In some embodiments, members with lower costs and/or faster recovery times would have their questions adjusted upward, thereby promoting questions related to outcomes.

In some embodiments, a member's mobile device (e.g. phone, tablet etc.) runs an application that is connected to the graph-based query prediction platform/system and passively monitors all audio inputs during the member's office visit. In addition to the audio being monitored, the application may have the ability to scan hard copy information given to the member during their visit. In some embodiments, the graph-based query prediction platform/system may analyze prior medical records, claims, etc.

In some embodiments, the application presents a list of suggested questions for the member to ask. In some embodiments, the member can select a question in the application as already asked or choose to skip a question. Either of these actions will refresh the list. In some embodiments, questions are presented in a ranked order.

For example, a member A is visiting their provider and is discussing their high cholesterol issue. Member A is vegan, and so questions about meat consumption are not relevant. In some embodiments, the graph-based query prediction platform/system may prompt the member A to ask about supplementation with certain vitamins (e.g. B12). These prompts are based on this member's similarity to other vegan members with high cholesterol.

In some embodiments, questions suggested to a member may also be shown to the provider to proactively address issues.

In some embodiments, a member may ask the provider a question which was not suggested by the graph-based query prediction platform/system. In some embodiments, the graph-based query prediction platform/system may recommend to the provider to explore the possibility of co-morbidities that are strongly associated with the question.

As such, various embodiments of the present disclosure provide a method to recommend personalized questions during or prior to the physician visit that are specific to the member, customized to their medical history, current conditions, personal details, based on the content of their interactions with their provider, and drawn from questions asked by other members with similar profiles and health conditions. Various embodiments of the present disclosure provide a method to monitor and analyze member/provider conversations and update the next-best-question in real time. Various embodiments of the present disclosure provide a method to inform a member of likely provider questions prior to the physician visit. Various embodiments of the present disclosure provide a method to recommend personalized questions to a provider during the visit.

Various embodiments of the present disclosure generates questions for the member to ask provider/physician based on the questions' propensity to influence the health outcome for similar member, generates questions by analyzing graph database of questions asked by similar members, and automatically learn from or analyze questions asked by a member and recommend questions for the member to ask.

As such, various embodiments of the present disclosure improve relevancy, applicability, and accuracy associated with predicted data that are generated in accordance with embodiments described herein. Additionally, the use of a graph data structure provides an efficient and scalable mechanism for performing question recommendations. This is especially true in the case of rare diseases when there may be a small amount of data to train a machine learning model on. In situations of very common medical conditions or in a very widely used system with many years of question feedback data, it would be possible to construct a non-graph-based question recommendation system using supervised or semi-supervised learning algorithms, for example.

### b. Definitions

In the present disclosure, the term “data object” refers to a data structure that represents, indicates, stores and/or comprises data and/or information. In some embodiments, a data object may be in the form of one or more regions in one or more data storage devices (such as, but not limited to, a computer-readable storage medium) that comprise one or more values (such as, but not limited to, one or more identifiers, one or more metadata, and/or the like). In some embodiments, an example data object may comprise or be associated with one or more identifiers, one or more metadata, and/or one or more other data objects.

In accordance with various embodiments of the present disclosure, data objects may be characterized based at least in part on the structure or format that data and/or information are organized in the data objects.

For example, a “graph data object” refers to a type of data object that utilizes one or more graph structures to represent, organize, and/or store data and/or information, and may, for example, utilize one or more graph structures to indicate, illustrate, and/or specify relationships between data and/or information. In some embodiments, an example graph data object may comprise one or more vertices and one or more edges that connect the one or more vertices, details of which are described herein.

In the present disclosure, the term “healthcare graph data object” may refer to a type of graph data object that may represent, organize, and/or store data and/or information related to healthcare, and may, for example, utilize one or more graph structures to indicate, illustrate, and/or specify relationships between data and/or information related to healthcare.

For example, in the healthcare context, a patient (also referred to as a “member” interchangeably herein) may conducted one or more visits to a healthcare provider (also referred to as a “provider” interchangeably herein) in settings such as, but not limited to, a doctor's office, a clinic, a pharmacy, a hospital, and/or the like to seek medical help, medical treatment, medical assistance, pharmacy prescriptions, and/or the like.

During these one or more visits, the member may ask one or more questions to the provider that are related to the reasons(s) for the one or more visits (for example, questions about the cause of the health conditions of concern, questions about treatments, questions about the effects of medications, and/or the like). In the present disclosure, the term “member query” refers to question(s) from a member to a provider associated with a member's visit to the provider. For example, a “historical member query” refers to question(s) that a member has asked a provider during the member's visit to the provider. As another example, a “predicted member query” refers to question(s) that are generated in accordance with various embodiments of the present disclosure and are recommended for a member to ask a provider during the member's visit to the provider, details of which are described herein.

Similarity, during these one or more visits, the provider may ask one or more questions to the member that are related to the reasons(s) for the one or more visits (for example, questions about the health conditions of concern to facilitate diagnosis, questions that may determine whether certain medication or treatment may be suitable for the member, and/or the like). In the present disclosure, the term “provider query” refers to question(s) from a provider to a member for a member's visit to the provider. For example, a “historical provider query” refers to question(s) that a provider has asked a member during the member's visit to the provider. As another example, a “predicted provider query” refers to question(s) that are generated in accordance with various embodiments of the present disclosure and are recommended for a provider to ask a member during the member's visit to the provider, details of which are described herein.

As described above, an example graph data object may comprise one or more vertices (and one or more edges that connect the vertices). For example, an example healthcare graph data object may comprise vertices such as, but not limited to, one or more member vertices, one or more attribute vertices, one or more visit vertices, one or more member query vertices, one or more provider query vertices, and/or the like. In some embodiments, one or more vertices in an example healthcare graph data object may be interconnected with one or more other vertices through one or more edges.

In the present disclosure, the terms “member vertex” or “member vertices” may refer to a point and/or a node in an example healthcare graph data object that corresponds to, indicates, and illustrates a member associated with the example graph-based query prediction platform/system illustrated above. As described above, the member may be a patient that has conducted or plans to conduct a visit to a provider. In the example graph-based query prediction platform/system, each member is assigned to a “member identifier,” and each member vertex is associated with a corresponding member identifier. In some embodiments, the “member identifier” uniquely identifies data and/or information stored in the example graph-based query prediction platform/system that is related to a member. For example, a member identifier may comprise American Standard Code for Information Interchange (ASCII) text, a pointer, a memory address, and the like.

In the present disclosure, the terms “historical member vertex” or “historical member vertices” may refer to a type of member vertex that is associated with a member who has conducted one or more visits to one or more providers. In some embodiments, an example graph-based query prediction platform/system may generate one or more predicted member query vertices associated with a member vertex (e.g. associated with a member Alex), and historical member vertex/vertices refer to member vertex/vertices that are associated with other members (e.g. associated with members other than Alex).

As an example, in accordance with various embodiments of the present disclosure, the example graph-based query prediction platform/system may generate one or more predicted member query vertices for a member Alex based on, such as but not limited to, vertices associated with a member Brandon, details of which are described herein. In this example, the member vertex associated with the user Alex is a current member vertex (or a member vertex for the simplicity of description). The member vertex associated with the user Brandon is a historical member vertex.

In the present disclosure, the terms “provider vertex” or “provider vertices” may refer to a point and/or a node in an example healthcare graph data object that corresponds to, indicates, and illustrates a provider associated with the example graph-based query prediction platform/system illustrated above. As described above, the provider may be, for example but not limited to, a doctor, a pharmacist, and/or the like who provides and/or will provide healthcare services to a member during the member's visit to the provider. In the example graph-based query prediction platform/system, each provider is assigned to a “provider identifier,” and each provider vertex is associated with a corresponding provider identifier. In some embodiments, the “provider identifier” uniquely identifies data and/or information stored in the example graph-based query prediction platform/system that is related to a provider. For example, a provider identifier may comprise ASCII text, a pointer, a memory address, and the like.

In the present disclosure, the terms “historical provider vertex” or “historical provider vertices” may refer to a type of provider vertex that is associated with a provider who has received one or more visits from one or more members, and/or associated with a provider who is not the provider for whom the example graph-based query prediction platform/system generates one or more predicted provider query vertices.

As an example, in accordance with various embodiments of the present disclosure, the example graph-based query prediction platform/system may generate one or more predicted provider query vertices for a provider Alex based on, such as but not limited to, vertices associated with a provider Brandon, details of which are described herein. In this example, the provider vertex associated with the user Alex is a current provider vertex (or a provider vertex for the simplicity of description). The provider vertex associated with the user Brandon is a historical provider vertex.

In the present disclosure, the terms “attribute vertex” or “attribute vertices” may refer to a point and/or a node in an example healthcare graph data object that corresponds to, indicates, and illustrates data and/or information of one or more attributes associated with a member. Examples of data and/or information of one or more attributes associated with the member may include, but not limited to, demographic data/information (such as, but not limited to, age, race, ethnicity, gender, sexual orientation, marital status, and/or the like), health history data/information (such as, but not limited to, allergies, prior illnesses, prior or current treatments, prior surgeries, prior immunizations, prior or current medications, results of physical exams and tests, and/or the like), symptom data/information (such as, but not limited to, heartache, headache, pain, feeling of fatigue, numbness, and/or the like), and/or the like.

In some embodiments, an example healthcare graph data object may comprise individualized attribute vertices for different demographic data/information, different health history data/information, and/or different symptom data/information. As an example, the example healthcare graph data object may comprise an attribute vertex for headache, an attribute vertex for feeling of fatigue, and/or the like. If a member vertex is connected to the attribute vertex for headache, it indicates that the member corresponding to the member vertex is experiencing headache.

In some embodiments, the terms “attribute vertex set” or “attribute vertices set” refers to a collection of zero or more attribute vertices. For example, an example attribute vertex set may comprise one attribute vertex from an example healthcare graph data object. As another example, an example attribute vertex set may comprise two attribute vertices, and so on.

In some embodiments, an example healthcare graph data object may comprise a plurality of attribute vertex sets. In some embodiments, an example attribute vertex in an example healthcare graph data object may be a part of one attribute vertex set. In some embodiments, an example attribute vertex in an example healthcare graph data object may be a part of multiple attribute vertex sets (e.g. an overlapping attribute vertex among the multiple attribute vertex sets).

For example, an example attribute vertex may be an overlapping attribute vertex in a first attribute vertex set that is associated with a first member vertex (e.g. attribute vertices that are connected to the first member vertex) and a second attribute vertex set that is associated with a second member vertex (e.g. attribute vertices that are connected to the second member vertex).

In the present disclosure, the terms “visit vertex” or “visit vertices” may refer to a point and/or a node in an example healthcare graph data object that corresponds to, indicates, and illustrates a visit by a member to a provider. As described above, the member may be a patient who has conducted or plans to conduct a visit to a provider (e.g., a doctor, a pharmacist, and/or the like). In the example graph-based query prediction platform/system, each visit is assigned to a “visit identifier,” and each visit vertex is associated with a corresponding visit identifier. In some embodiments, the “visit identifier” uniquely identifies data and/or information stored in the example graph-based query prediction platform/system that is related to a visit. For example, a visit identifier may comprise ASCII text, a pointer, a memory address, and the like.

In the present disclosure, the terms “historical visit vertex” or “historical visit vertices” may refer to a point and/or a node in an example healthcare graph data object that is associated with one or more visits to one or more providers that have already been conducted by one or more members. As an example, in accordance with various embodiments of the present disclosure, the example graph-based query prediction platform/system may generate one or more predicted member query vertices for a member Alex based on, such as but not limited to, visit vertices associated with a member Brandon (that are based on Brandon's past visits), details of which are described herein. In this example, the visit vertex associated with the user Alex is a current visit vertex (or a visit vertex for the simplicity of description). The visit vertex associated with the user Brandon is a historical visit vertex.

In the present disclosure, the terms “member query vertex” or “member query vertices” may refer to a point and/or a node in an example healthcare graph data object that corresponds to, indicates, and illustrates data and/or information associated with one or more question(s) from a member to a provider for a member's visit to the provider (e.g. one or more member queries). For example, an example member query vertex may comprise data and/or information in the form of questions that a member has asked and/or is recommended to ask a provider during the member's visit to the provider. Examples of questions may include, but are not limited to, questions about the cause of the health conditions of concern, questions about treatments, questions about the effects of medications, and/or the like.

In some embodiments, an example member query vertex may be an example historical member query vertex or an example predicted member query vertex.

In the present disclosure, the terms “historical member query vertex” or “historical member query vertices” may refer to a type of member query vertex that corresponds to, indicates, and illustrates data and/or information associated with one or more question(s) that a member has asked a provider in the past (e.g. one or more historical queries).

In some embodiments, an example graph-based query prediction platform/system may generate the initial historical member query vertex or initial historical member query vertices through a bootstrapping process and/or based on publicly available databases.

For example, through the bootstrapping process, the graph-based query prediction platform/system may collect questions that real members have asked or are currently asking their providers. For example, the graph-based query prediction platform/system may implement a software application (e.g. a third-party application of the graph-based query prediction platform/system, a first-party application of the graph-based query prediction platform/system) that can be executed on a computing entity to collect questions asked by members during visits. Additionally, or alternatively, the graph-based query prediction platform/system may implement an Internet-of-Thing (IoT) device that may monitor conversations during a member's visit to the provider. Additionally, or alternatively, the graph-based query prediction platform/system may collect questions through other means. In some embodiments, the bootstrapping process may continue until enough training data is collected.

Additionally, or alternatively, an example graph-based query prediction platform/system may utilize question databases from, such as but not limited to, Oak Street Health, Cleveland Clinic, Conway Medical Center, and/or the like. Additionally, or alternatively, an example graph-based query prediction platform/system may use databases such as PubMed from the University of Canberra that are generally used for building evidence for clinical decision support systems, while many of those types of questions would be relevant to member inquiries of doctors. Additionally, or alternatively, search logs of members that may include search queries provided to a computing entity by the member may also be used as a data source for the initial bootstrapping process. While some of those sources are not lengthy and by themselves may not be enough to bootstrap the example graph-based query prediction platform/system, they can be sufficient in combination.

It is noted that various embodiments of the present disclosure may not need to have access to a huge public database for the initial bootstrapping process. For example, as various embodiments of the present disclosure are implemented, questions asked by members during the visits are collected, and an example graph-based query prediction platform/system may generate historical member query vertices accordingly.

In the present disclosure, the term “predicted member query vertex” or “predicted member query vertices” may refer to a type of member query vertex that corresponds to, indicates, and illustrates data and/or information associated with one or more question(s) that are determined/generated by an example graph-based query prediction platform/system and/or are recommended to a member for the member to ask a provider during the member's visit to the provider. In some embodiments, the graph-based query prediction platform/system may generate a predicted member query vertex based at least in part on the healthcare graph data object, details of which are described herein.

In the present disclosure, the terms “provider query vertex” or “provider query vertices” may refer to a point and/or a node in an example healthcare graph data object that corresponds to, indicates, and illustrates data and/or information associated with one or more question(s) from a provider to a member for a member's visit to the provider. For example, an example provider query vertex may comprise data and/or information in the form of questions that a provider has asked and/or is recommended to ask a member during the member's visit to the provider. Examples of questions may include, but are not limited to, questions about the health conditions of concern to facilitate diagnosis, questions that may determine whether certain medication or treatment may be suitable for the member, and/or the like.

In some embodiments, an example provider query vertex may be an example historical provider query vertex or an example predicted provider query vertex.

In the present disclosure, the terms “historical provider query vertex” or “historical provider query vertices” may refer to a type of provider query vertex that corresponds to, indicates, and illustrates data and/or information associated with one or more question(s) that a provider has asked a member in the past. In some embodiments, an example graph-based query prediction platform/system may generate the initial historical provider query vertex or initial historical provider query vertices through a bootstrapping process and/or based on publicly available databases, similar to those described above in connection with at least generating the initial historical member query vertex or initial historical member query vertices.

In the present disclosure, the term “predicted provider query vertex” or “predicted provider query vertices” may refer to a type of provider query vertex that corresponds to, indicates, and illustrates data and/or information associated with one or more question(s) that are determined/generated by an example graph-based query prediction platform/system and/or are recommended to a provider for the provider to ask the member during the member's visit to the provider. In some embodiments, the graph-based query prediction platform/system may generate a predicted provider query vertex based at least in part on the healthcare graph data object, details of which are described herein.

As described above, an example graph data object may comprise one or more edges. In the present disclosure, the term “edge” may refer to a line or a connector that connects two or more vertices. In some embodiments, an edge in an example healthcare graph data object indicates, illustrates, and/or specifies relationships between vertices.

For example, the term “member-query edge” may refer to one or more edges that connect an example member vertex and an example member query vertex in the healthcare graph data object. As another example, the term “member-visit edge” may refer to one or more edges that connect an example member vertex and an example visit vertex in the healthcare graph data object. As another example, the term “visit-query edge” may refer to one or more edges that connect an example visit vertex to an example member query vertex and/or an example provider query vertex in the healthcare graph data object.

In the present disclosure, an example edge in an example healthcare graph data object may be associated with an “edge weight.” In some embodiments, the term “edge weight” refers to a measure or a parameter in the healthcare graph data object that may quantitatively or qualitatively indicate, illustrate, and/or specify one or more characteristics or properties of the relationship(s) between the vertices that are connected by the edge.

For example, an example member vertex may be connected to a plurality of member query vertices through a plurality of member-query edges. In this example, each of the plurality of member-query edges may have a corresponding “member-query edge weight” that indicates the priority of the member query vertices relative to the example member vertex. For example, an example member query vertex that is connected to the example member vertex through a member-query edge having a higher member-query edge weight is prioritized over (e.g. more relevant than) another example member query vertex that is connected to the example member vertex through a member-query edge having a lower member-query edge weight.

Additionally, or alternatively, an example member vertex may be connected to a plurality of visit vertices through a plurality of member-visit edges. In this example, each of the plurality of member-visit edges may have a corresponding “member-visit edge weight” that indicates the priority of the visit vertices relative to the example member vertex. For example, an example visit vertex that is connected to the example member vertex through a member-visit edge having a higher member-visit edge weight is prioritized over (e.g. more relevant than) another example visit vertex that is connected to the example member vertex through a member-visit edge having a lower member-visit edge weight.

Additionally, or alternatively, an example visit vertex may be connected to a plurality of member query vertices through a plurality of visit-query edges. In this example, each of the plurality of visit-query edges may have a corresponding “visit-query edge weight” that indicates the priority of the member query vertices relative to the example visit vertex. For example, an example member query vertex that is connected to the example visit vertex through a visit-query edge having a higher visit-query edge weight is prioritized over (e.g. more relevant than) another example member query vertex that is connected to the example visit vertex through a visit-query edge having a lower member-query edge weight.

In accordance with various embodiments of the present disclosure, edge weight (such as, but not limited to, member-query edge weights, member-visit edge weights, visit-query edge weights, and/or the like) may be determined based on one or more healthcare measures, such as, but not limited to, healthcare outcome measures and/or healthcare cost measures.

In the present disclosure, the term “healthcare outcome measure” may refer to a measure or a parameter that is associated with a member identifier (corresponding to a member) and/or a visit identifier (corresponding to a visit), and may quantitatively or qualitatively indicate, illustrate, and/or specify the quality, effectiveness and/or successfulness of a member's visit to a provider. For example, the healthcare outcome measure may quantitatively or qualitatively indicate whether (and the level as to which) the provider addresses or solves the member's health concerns (e.g. the reasons for the visit) during the visit. Additionally, or alternatively, the healthcare outcome measure may quantitatively or qualitatively indicate whether (and the level as to which) the solution prescribed by the provider (e.g. medications, treatments, etc.) addresses or solves the member's health concerns (e.g. the reasons for the visit). Additionally, or alternatively, the healthcare outcome measure may quantitatively or qualitatively indicate how quickly the member recovers from the health concerns (that are the reasons for the visit) after the visit. Additionally, or alternatively, the healthcare outcome measure may quantitatively or qualitatively indicate a satisfaction level of the visit as indicated by the member.

In the present disclosure, the term “healthcare cost measure” may refer to a measure or a parameter that is associated with a member identifier (corresponding to a member) and/or a visit identifier (corresponding to a visit), and may quantitatively or qualitatively indicate, illustrate, and/or specify a healthcare cost associated with the member and/or the visit. For example, the healthcare cost measure may quantitatively or qualitatively indicate, illustrate, and/or specify costs associated with the medication(s) that the provider prescribed to address the member's health concern (e.g. the reasons for the visit). Additionally, or alternatively, the healthcare cost measure may quantitatively or qualitatively indicate, illustrate, and/or specify costs associated with the procedure(s) that the provider conducted on the patient during the visit to address the member's health concern (e.g. the reasons for the visit). Additionally, or alternatively, the healthcare cost measure may quantitatively or qualitatively indicate, illustrate, and/or specify costs associated with the service(s) that the provider rendered to address the member's health concern (e.g. the reasons for the visit) during the member's visit to the provider.

As described above, an example graph data object may comprise vertices and edges that connect the vertices. In some embodiments, an example graph data object may comprise one or more subgraphs. In the present disclosure, the term “subgraph” refers to a portion of a healthcare graph data object that comprise at least one of a vertex in the healthcare graph data object, and one or more other vertices that are directly and/or indirectly connected to the vertex through one or more edges, if any.

For example, the term “member subgraph” may refer to a type of subgraph that comprises a member vertex and one or more other vertices that are directly (and/or indirectly) connected to the member vertex such as, but not limited to, one or more attribute vertices. In the present disclosure, the term “historical member subgraph” may refer to a type of member subgraph that comprises a historical member vertex and one or more other vertices (such as attribute vertices) that are directly (and/or indirectly) connected to the historical member vertex.

For example, the term “provider subgraph” may refer to a type of subgraph that comprises a provider vertex and one or more other vertices that are directly (and/or indirectly) connected to the provider vertex such as, but not limited to, one or more attribute vertices. In the present disclosure, the term “historical provider subgraph” may refer to a type of provider subgraph that comprises a historical provider vertex and one or more other vertices (such as attribute vertices) that are connected to the historical provider vertex.

In accordance with various embodiments of the present disclosure, data objects may be characterized and/or categorized based at least in part on the content of data and/or information that the data objects comprise, represent, and/or store.

For example, an electronic health record data object or an EHR data object may refer to a type of data object that comprises, represents, and/or stores health record data and/or information associated with a member. For example, an example electronic health record data object may comprise demographic information and/or health history information associated with the member. Examples of demographic information may include, but are not limited to, age, race, ethnicity, gender, sexual orientation, marital status, and/or the like. Examples of health history information may include, but not limited to, allergies, prior illnesses, prior or current treatments, prior surgeries, prior immunizations, prior or current medications, results of physical exams and tests, and/or the like. In some embodiments, an example electronic health record data object comprises data and/or information that a patient may provide during a visit to a provider (e.g. collected for a medical chart). Additionally, or alternatively, the electronic health record data object may comprise symptom information that describes the symptoms associated with a member.

In the present disclosure, the term “member data object” may refer to a type of data object that comprises, represents, and/or stores data and/or information associated with a member. In some embodiments, an example member data object may comprise and/or be associated with one or more data objects, such as, but not limited to, one or more member demographics data objects, one or more member history data objects, one or more member symptom data objects, and/or one or more member supplemental data objects.

In the present disclosure, the term “member demographics data object” may refer to a type of member data object that comprises, represents, and/or stores demographic information associated with the member that may include, but are not limited to, age, race, ethnicity, gender, sexual orientation, marital status, and/or the like.

In the present disclosure, the term “member history data object” may refer to a type of member data object that comprises, represents, and/or stores health history information associated with the member that may include, but are not limited to, allergies, prior illnesses, prior or current treatments, prior surgeries, prior immunizations, prior or current medications, results of physical exams and tests, and/or the like.

In the present disclosure, the term “member symptom data object” may refer to a type of member data object that comprises, represents, and/or stores data and/or information of symptoms associated with the member that may include, but are not limited to, physical symptoms (such as aches, pains, dizziness), psychological symptoms (such as confusion, mood swings), visual symptoms (such as wound, cold sores), audio symptoms (such as change in speech pattern), and/or the like.

In the present disclosure, the term “member supplemental data object” may refer to a type of member data object that comprises, represents, and/or stores data and/or information that is collected during the member's visit to the provider, details of which are described herein.

In the present disclosure, the term “natural language processing model” refers to a software computer algorithm (such as, but not limited to, computer programs, machine learning models, artificial intelligence models, and/or the like) (and, in some embodiments, associated hardware) that processes, analyzes, generates, integrates, summarizes, translates, and/or predicts (and/or the like) natural language data (such as, but not limited to, textual inputs).

While the description above provides an example of a natural language processing model, it is noted that the scope of the present disclosure is not limited to the description above. In some embodiments, an example natural language processing model may be applied to other types of data.

In the present disclosure, the term “graph-based machine learning model” may refer to a software computer program (and, in some embodiments, associated hardware) that is trained to process, analyze, generate, integrate, summarize, translate, and/or predict one or more output datasets (such as metrics) based at least in part on one or more graph data objects (such as a healthcare graph data object). For example, an example machine learning model may be trained to analyze a healthcare graph data object and generate one or more resemblance metrics and/or one or more prioritization metrics. Examples of graph-based machine learning models may include, but are not limited to, artificial neural networks, graph-neural networks, graph neural networks, and/or the like.

In the present disclosure, the term “resemblance metrics” may refer to a measure or a parameter that may quantitatively or qualitatively indicate, illustrate, and/or specify how similar two or more vertices and/or two or more subgraphs are in a healthcare graph data object. For example, an example resemblance metrics may indicate a similarity measure between a historical member vertex and a member vertex. Additionally, or alternatively, an example resemblance metrics may indicate a similarity measure between a historical member subgraph and a member subgraph. In the present disclosure, the term “cosine similarity measures” may refer to a type of similarity measure that is defined based on the cosine of the angle between the two or more vertices and/or two or more subgraphs.

In the present disclosure, the term “prioritization metrics” may refer to a measure or a parameter that may quantitatively or qualitatively indicate, illustrate, and/or specify the priority between and/or among two or more vertices. For example, prioritization metrics may indicate priorities between and/or among predicted member query vertices generated in accordance with various embodiments of the present disclosure. As described further in detail herein, predicted member query indications may be rendered on a graphic user interface based on the predicted member query vertices, and an order or a ranking of the predicted member query indications may be determined corresponding to the associated resemblance metrics and/or the associated prioritization metrics.

As described above, an example graph-based machine learning model may be trained to generate resemblance metrics and/or prioritization metrics.

In the present disclosure, the term “training healthcare graph data object” may refer to a type of healthcare graph data object that can be provided to a graph-based machine learning model for training. For example, an example training healthcare graph data object may comprise one or more training member vertices and/or one or more training member subgraphs, and the resemblance metrics between the one or more training member vertices and/or between the one or more training member subgraphs are known (also referred to as training resemblance metrics). Additionally, or alternatively, an example training healthcare graph data object may comprise one or more training edges associated with known prioritization metrics (also referred to as training prioritization metrics).

In the present disclosure, the term “prediction-based action” may refer to one or more computer operations based at least in part on one or more predicted member query vertices. For example, an example prediction-based action may be rendering/updating a graphic user interface based at least in part on the predicted member query vertices.

In the present disclosure, the term “graphic user interface” may refer to a user interface that enables a user (for example, a member) to interact with a computing entity (e.g. providing inputs to the computing entity and receiving outputs from the computing entity). For example, an example graphic user interface may comprise one or more graphic icons and/or elements, and can be rendered on a display of the computing entity. Examples of graphic user interface are illustrated herein, including, but not limited to, at least FIG. 15, FIG. 16, FIG. 17, FIG. 18, and FIG. 19.

In the present disclosure, the term “predicted member query indication” or “predicted member query indications” may refer to a rendering of a predicted member query vertex on a graphic user interface. For example, as described above, an example predicted member query vertex may include data and/or information associated with one or more question(s) that are determined/generated by an example graph-based query prediction platform/system and/or are recommended to a member for the member to ask a provider during the member's visit to the provider. As such, an example predicted member query indication on a graphic user interface may display the one or more question(s) that are determined/generated by an example graph-based query prediction platform/system.

In the present disclosure, the term “predicted provider query indication” or “predicted provider query indications” may refer to a rendering of a predicted provider query vertex on a graphic user interface. For example, as described above, an example predicted provider query vertex may include data and/or information associated with one or more question(s) that are determined/generated by an example graph-based query prediction platform/system and/or are recommended to a provider for the provider to ask a member during the member's visit to the provider. As such, an example predicted provider query indication on a graphic user interface may display the one or more question(s) that are determined/generated by an example graph-based query prediction platform/system.

In the present disclosure, the term “user input” may refer to data and/or information that is sent to a computing entity. For example, the term “audio input” may refer to a type of user input that is in an audio form (e.g. member voices and/or conversations between a member and a provider as detected by a microphone of the computing entity, and/or the like). In the present disclosure, the term “visual input” may refer to a type of user input that is in a visual form (e.g. one or more pictures associated with a member's visit that are captured by a camera of the computing entity during the member's visit to the provider). In the present disclosure, the term “textual inputs” may refer to a type of user input that is in a textual form (e.g. texts that are written by the member, converted from audio input through a speech-to-text algorithm, and/or the like). In the present disclosure, the term “supplemental data inputs” may refer to a type of user input that is collected during the member's visit to the provider.

Additionally, or alternatively, a member may provide various user inputs with regards to predicted member query indications displayed on a graphic user interface as described above. For example, the member may provide a user input indicating a user disinterest of a predicted member query indication that corresponds to a question generated by the graph-based query prediction platform/system, but is not deemed to be appropriate or suitable by the member. Additionally, or alternatively, the member may provide a user input indicating a user interest of a predicted member query indication that corresponds to a question generated by the graph-based query prediction platform/system and is deemed to be appropriate or suitable by the member.

In accordance with various embodiments of the present disclosure, a member may provide various indications to the graph-based query prediction platform/system through, for example but not limited to, an example client computing entity as described above.

For example, the term “actual member visit indication” may refer to an indication provided by a member that the member is currently in a visit to a provider. The term “anticipatory member visit indication” may refer to an indication provided by a member that the member plans to conduct a visit to a provider in the future.

### c. Exemplary Techniques for Generating Predicted Query Vertices

As described above, there are technical challenges, deficiencies and problems associated with database systems, and various example embodiments of the present disclosure overcome such challenges. For example, referring now to FIG. 4, an example method 400 of generating predicted member query vertices in accordance with embodiments of the present disclosure is illustrated.

For example, the example method 400 may connect one or more attribute vertices associated with a member vertex in a healthcare graph data object, determine one or more historical member vertices from the healthcare graph data object, determine one or more historical member query vertices from the healthcare graph data object, generate one or more predicted member query vertices in the healthcare graph data object, and perform one or more prediction-based actions based at least in part on the one or more predicted member query vertices. As such, the example method 400 may, for example but not limited to, improve relevancy, applicability, and accuracy associated with the predicted data.

As shown in FIG. 4, the example method 400 starts at step/operation 402. Subsequent to and/or in response to step/operation 402, the example method 400 proceeds to step/operation 404. At step/operation 404, a computing entity (such as the query prediction computing entity 105 described above in connection with FIG. 1 and FIG. 2) may include means (such as the processing element 205 of the query prediction computing entity 105 described above in connection with FIG. 2) to generate one or more edges connecting one or more attribute vertices to a member vertex in a healthcare graph data object.

As described above, the attribute vertices may refer to points and/or nodes in an example healthcare graph data object that correspond to, indicate, and illustrate data and/or information of attributes of members such as, but not limited to, demographic data/information (such as, but not limited to, age, race, ethnicity, gender, sexual orientation, marital status, and/or the like), health history data/information (such as, but not limited to, allergies, prior illnesses, prior or current treatments, prior surgeries, prior immunizations, prior or current medications, results of physical exams and tests, and/or the like), symptom data/information (such as, but not limited to, heartache, headache, pain, feeling of fatigue, numbness, and/or the like), and/or the like. In some embodiments, the computing entity may connect one or more attribute vertices to a member vertex associated with a member identifier in a healthcare graph data object based at least in part on one or more member data objects that are associated with the member identifier.

For example, the healthcare graph data object may comprise at least one of a member demographics data object associated with a member identifier, a member history data object associated with a member identifier, and/or a member symptom data object associated with a member identifier. As described above, the example member demographics data object may comprise, represent, and/or store demographic information associated with the member. The member history data object may comprise, represent, and/or store health history information associated with the member. The member symptom data object may comprise, represent, and/or store data and/or information of symptoms associated with the member.

As an example, a member Alex of the graph-based query prediction platform/system may plan to conduct a visit to a provider or is conducting a visit to the provider for his symptoms related to pain in the leg and limping. In this example, the computing entity may determine a member vertex in the healthcare graph data object that is associated with a member identifier corresponding to Alex. In this example, the computing entity may retrieve one or more member data objects associated with Alex from a database that is internal to or external to the graph-based query prediction platform/system.

For example, the one or more member data objects associated with Alex may include a member demographics data object comprising data/information indicating that Alex is a 67 year old male who lives in Houston, Tex. In this example, the computing entity may generate an edge connecting the member vertex that represents Alex with an attribute vertex that represents “67 years old,” may generate an edge connecting the member vertex that represents Alex with an attribute vertex that represents “male,” and may generate an edge connecting the member vertex that represents Alex with an attribute vertex that represents “Houston, Tex.”

Additionally, or alternatively, the one or more member data objects associated with Alex may include a member history data object comprising data/information indicating that Alex has Type II diabetes, hypertension and total knee replacement. In this example, the computing entity may generate an edge connecting the member vertex that represents Alex with an attribute vertex that represents “Type II diabetes,” may generate an edge connecting the member vertex that represents Alex with an attribute vertex that represents “hypertension,” and may generate an edge connecting the member vertex that represents Alex with an attribute vertex that represents “total knee replacement.”

Additionally, or alternatively, the one or more member data objects associated with Alex may include a member symptom data object comprising data/information indicating that Alex has pain in the leg and limping. In this example, the computing entity may generate an edge connecting the member vertex that represents Alex with an attribute vertex that represents “pain in the leg” and may generate an edge connecting the member vertex that represents Alex with an attribute vertex that represents “limping.”

In some embodiments, the computing entity may determine that the member is associated with one or more attributes that do not have corresponding attribute vertices in the healthcare graph data object. In these embodiments, the computing entity may generate the one or more attribute vertices corresponding to these attributes, and connect the member vertex to these one or more attribute vertices.

Continuing from the example above, if the computing entity determines that Alex has Type II diabetes, but there is no attribute vertex in the healthcare graph data object that corresponds to “Type II diabetes,” the computing entity may generate an attribute vertex for Type II diabetes, and may connect the member vertex corresponding to Alex with the attribute vertex that corresponds to Type II diabetes.

Referring back to FIG. 4, subsequent to and/or in response to step/operation 404, the example method 400 proceeds to step/operation 406. At step/operation 406, a computing entity (such as the query prediction computing entity 105 described above in connection with FIG. 1 and FIG. 2) may include means (such as the processing element 205 of the query prediction computing entity 105 described above in connection with FIG. 2) to determine one or more historical member vertices from the healthcare graph data object.

As described above, historical member vertices refer to member vertices that are associated with members who have conducted one or more visits to one or more providers. Continuing from the Alex example, the computing entity may determine/select one or more member vertices that are associated with members other than Alex as one or more historical member vertices.

In some embodiments, the computing entity may determine/select one or more historical member vertices based at least in part on the one or more attribute vertices that are connected to the member vertex at step/operation 404. For example, each of the one or more historical member vertices may be associated with and/or connected to one or more attribute vertices, and the computing entity may determine, select and/or rank one or more historical member vertices at least according to one or more resemblance metrics associated with the one or more historical member vertices and relative to the member vertex based on the one or more attribute vertices connected to the member vertex and the one or more attribute vertices connected to the historical member vertices, details of which are described in connection with at least FIG. 7 to FIG. 9. In some embodiments, the computing entity may determine one or more historical member vertices from the healthcare graph data object using at least one graph-based machine learning model, details of which are described in connection with at least FIG. 7 to FIG. 9.

Continuing from the Alex example above, the example healthcare graph data object may comprise a member vertex that corresponds to a member Brandon and connected to a first set of attribute vertices and a member vertex that corresponds to a member Cindy and connected to a second set of attribute vertices. As an example, the computing entity may determine a resemblance metrics associated with the member vertex that corresponds to Brandon and relative to the member vertex that corresponds to Alex (e.g. based on the first set of attribute vertices and the attribute vertices connected to the member vertex that corresponds to Alex), and may determine a resemblance metrics associated with the member vertex that corresponds to Cindy and relative to the member vertex that corresponds to Alex (e.g. based on the second set of attribute vertices and the attribute vertices connected to the member vertex that corresponds to Alex). In this example, the computing entity may determine, select, and/or rank one of the member vertices that corresponds to Brandon or the member vertex that corresponds to Cindy as a historical member vertex based on the resemblance metrics.

Referring back to FIG. 4, subsequent to and/or in response to step/operation 406, the example method 400 proceeds to step/operation 408. At step/operation 408, a computing entity (such as the query prediction computing entity 105 described above in connection with FIG. 1 and FIG. 2) may include means (such as the processing element 205 of the query prediction computing entity 105 described above in connection with FIG. 2) to determine one or more historical member query vertices from the healthcare graph data object.

As described above, historical member query vertices may correspond to, indicate, and illustrate one or more question(s) that a member has asked a provider in the past. In some embodiments, the computing entity may determine, select, and/or rank one or more historical member query vertices from the healthcare graph data object based at least in part on the one or more historical member vertices. For example, the computing entity may determine, select, and/or rank the one or more historical member query vertices that are connected to and/or associated with the historical member vertices determined at step/operation 406.

Continuing from the Alex example above, the computing entity may determine the member vertex corresponding to Brandon as a historical member vertex at step/operation 406. In this example, the computing entity may select the one or more historical member query vertices that are connected to and/or associated with the member vertex corresponding to Brandon (e.g. vertices comprising data and/or information associated with questions that Brandon has asked in the past) at step/operation 408.

In some embodiments, an example historical member vertex may be connected to and/or associated with a plurality of historical member query vertices. In some embodiments, the computing entity may determine, select, and/or rank one or more historical member query vertices from the healthcare graph data object at least according to one or more prioritization metrics associated with the one or more historical member query vertices, details of which are described in connection with at least FIG. 10 to FIG. 13. In some embodiments, the computing entity may determine, select, and/or rank one or more historical member query vertices from the healthcare graph data object using the at least one graph-based machine learning model, details of which are described in connection with at least FIG. 10 to FIG. 13.

Continuing from the Alex example above, the member vertex corresponding to Brandon may be connected to a first historical member query vertex, a second historical member query vertex, and a third historical member query vertex. In some embodiments, the computing entity may determine, select, and/or rank one or more historical member query vertices from the first historical member query vertex, the second historical member query vertex, and the third historical member query vertex based on their corresponding prioritization metrics (and/or resemblance metrics), details of which are described herein.

Referring back to FIG. 4, subsequent to and/or in response to step/operation 408, the example method 400 proceeds to step/operation 410. At step/operation 410, a computing entity (such as the query prediction computing entity 105 described above in connection with FIG. 1 and FIG. 2) may include means (such as the processing element 205 of the query prediction computing entity 105 described above in connection with FIG. 2) to generate one or more predicted member query vertices in the healthcare graph data object.

In some embodiments, the computing entity may generate the one or more predicted member query vertices in the healthcare graph data object based at least in part on the one or more historical member query vertices that are determined and/or selected at step/operation 408 above. For example, the one or more predicted member query vertices may be the same or similar as the one or more historical member query vertices that are determined and/or selected at step/operation 408 above.

Continuing from the Alex example, the computing entity may determine/select a historical member query vertex that is associated with the member vertex corresponding to Brandon and represents a question “is this because of nerve compression, muscle or bone injury?” In this example, the computing entity may generate a predicted member query vertex that represents the question “is this because of nerve compression, muscle or bone injury?”

In some embodiments, the computing entity may generate one or more edges connecting the one or more predicted member query vertices generated at step/operation 410 to the member vertex. Continuing from the Alex example above, the computing entity may generate an edge that connects the predicted member query vertex that represents the question “is this because of nerve compression, muscle or bone injury?” to the member vertex that corresponds to Alex.

Referring back to FIG. 4, subsequent to and/or in response to step/operation 410, the example method 400 proceeds to step/operation 412. At step/operation 412, a computing entity (such as the query prediction computing entity 105 described above in connection with FIG. 1 and FIG. 2) may include means (such as the processing element 205 of the query prediction computing entity 105 described above in connection with FIG. 2) to perform one or more prediction-based actions based at least in part on the one or more predicted member query vertices.

For example, the computing entity may render/update or cause rendering/updating a graphic user interface to include one or more predicted member query indications that are generated based on the predicted member query vertices. As described above, an example predicted member query vertex may include data and/or information associated with one or more question(s) that are determined/generated as recommended for the member to ask a provider during the member's visit to the provider. As such, an example predicted member query indication on a graphic user interface may display the recommended one or more question(s). Through the graphic user interface, the member is notified of the recommended questions so that the member can ask the provider such questions during the visit.

While the description above provides an example of prediction-based action, it is noted that the scope of the present disclosure is not limited to the description above. In some examples, an example prediction-based action may comprise one or more additional and/or alternative operations.

Referring back to FIG. 4, subsequent to and/or in response to step/operation 412, the example method 400 proceeds to step/operation 414 and ends.

While the description above provides some examples in accordance with example embodiments of the present disclosure, it is noted that the scope of the present disclosure is not limited to the examples above. For example, in accordance with various embodiments of the present disclosure, a computing entity (such as the query prediction computing entity 105 described above in connection with FIG. 1 and FIG. 2) may include means (such as the processing element 205 of the query prediction computing entity 105 described above in connection with FIG. 2) to generate predicted provider query vertices.

As described above, predicted provider query vertices may indicate and illustrate data and/or information associated with one or more question(s) that are determined/generated by the example graph-based query prediction platform/system and/or are recommended to a provider for the provider to ask the member during the member's visit to the provider.

For example, to generate predicted provider query vertices, the computing entity may generate one or more edges connecting one or more attribute vertices associated with a member vertex in a healthcare graph data object, similar to those described in connection with at least step/operation 404 of FIG. 4 and at least in connection with FIG. 5 to FIG. 6B. For example, the computing entity may generate the one or more edges based at least in part on the one or more member data objects comprising at least one of a member demographics data object, a member history data object, or a member symptom data object, similar to those described in connection with at least step/operation 404 of FIG. 4 and at least in connection with FIG. 5 to FIG. 6B.

Continuing from this example, the computing entity may determine one or more historical member vertices from the healthcare graph data object, similar to those described in connection with at least step/operation 406 of FIG. 4 and at least in connection with FIG. 7 to FIG. 9. For example, the computing entity may determine/select the one or more historical member vertices using at least one graph-based machine learning model and based at least in part on the one or more attribute vertices, similar to those described in connection with at least step/operation 406 of FIG. 4 and at least in connection with FIG. 7 to FIG. 9.

Continuing from this example, the computing entity may determine one or more historical provider query vertices from the healthcare graph data object. As described above, historical provider query vertices may correspond to, indicate, and illustrate one or more question(s) that a provider has asked a member in the past. In some embodiments, the computing entity may determine/select one or more historical provider query vertices from the healthcare graph data object based at least in part on the one or more historical member vertices. For example, the computing entity may determine/select the one or more historical provider query vertices that are connected to and/or associated with the historical member vertices.

In some embodiments, an example historical member vertex may be connected to and/or associated with a plurality of historical provider query vertices. In some embodiments, the computing entity may determine one or more historical provider query vertices from the healthcare graph data object at least according to one or more prioritization metrics associated with the one or more historical provider query vertices, details of which are similar to those described in connection with at least FIG. 10 to FIG. 13. In some embodiments, the computing entity may determine one or more historical provider query vertices from the healthcare graph data object using the at least one graph-based machine learning model, details of which are similar to those described in connection with at least FIG. 10 to FIG. 13.

Continuing from this example, the computing entity may generate one or more predicted provider query vertices in the healthcare graph data object based at least in part on the historical provider query vertices determined above. For example, the one or more predicted provider query vertices may be the same or similar as the one or more historical provider query vertices that are determined and/or selected above.

Continuing from this example, the computing entity may perform one or more prediction-based actions based at least in part on the one or more predicted provider query vertices. For example, the computing entity may render/update or cause the rendering/updating a graphic user interface to include one or more predicted provider query indications that are generated based on the predicted provider query vertices. As described above, an example predicted provider query indication on a graphic user interface may display the recommended one or more question(s) for the provider. Through the graphic user interface, the provider is notified of the recommended questions so that the provider can ask the member such questions during the member's visit to the provider.

### d. Exemplary Techniques for Connecting Attribute Vertices to Member Vertices

As described above, there are technical challenges, deficiencies and problems associated with database systems, and various example embodiments of the present disclosure overcome such challenges. For example, referring now to FIG. 5, an example method 500 of generating member demographics data objects and the member history data objects for the member data objects in accordance with embodiments of the present disclosure is illustrated.

For example, the example method 500 may retrieve at least one electronic health record data object associated with the member identifier, generate the member demographics data object based at least in part on the demographic information from the at least one electronic health record data object, and generate the member history data object based at least in part on the health history information from the at least one electronic health record data object. As such, the example method 500 may, for example but not limited to, improve relevancy, applicability, and accuracy associated with the predicted data.

As shown in FIG. 5, the example method 500 starts at step/operation 501. Subsequent to and/or in response to step/operation 501, the example method 500 proceeds to step/operation 503. At step/operation 503, a computing entity (such as the query prediction computing entity 105 described above in connection with FIG. 1 and FIG. 2) may include means (such as the processing element 205 of the query prediction computing entity 105 described above in connection with FIG. 2) to retrieve at least one electronic health record data object associated with the member identifier.

As described above, the electronic health record data object may comprise, represent, and/or store health record data and/or information associated with a member. In some embodiments, the at least one electronic health record data object may be stored in a database that is internal to the graph-based query prediction platform/system, and the computing entity may retrieve the electronic health record data object based on the member identifier. In some embodiments, the at least one electronic health record data object may be stored in a database that is external to the graph-based query prediction platform/system, and the computing entity may retrieve the electronic health record data object based on the member identifier.

In some embodiments, the at least one electronic health record data object comprises demographic information and health history information associated with the member identifier. As described above, examples of demographic information may include, but are not limited to, age, race, ethnicity, gender, sexual orientation, marital status, and/or the like. Examples of health history information may include, but not limited to, allergies, prior illnesses, prior or current treatments, prior surgeries, prior immunizations, prior or current medications, results of physical exams and tests, and/or the like.

As an example, the computing entity may retrieve an electronic health record data object that is associated with a member Alex. In this example, the electronic health record data object may comprise demographic information indicating that Alex is a 67 year old male who lives in Houston, Tex. The electronic health record data object may comprise member history data object indicating that Alex has Type II diabetes, hypertension and total knee replacement.

While the description above provides examples of information from an example electronic health record data object, it is noted that the scope of the present disclosure is not limited to the description above. In some examples, an example electronic health record data object may comprise additional and/or alternative information, such as, but not limited to, symptom information associated with the member. Continuing from the Alex example above, the electronic health record data object may comprise symptom information indicating that Alex is having pain in the leg and limping.

Referring back to FIG. 5, subsequent to and/or in response to step/operation 503, the example method 500 proceeds to step/operation 505. At step/operation 505, a computing entity (such as the query prediction computing entity 105 described above in connection with FIG. 1 and FIG. 2) may include means (such as the processing element 205 of the query prediction computing entity 105 described above in connection with FIG. 2) to generate the member demographics data object based at least in part on the demographic information from the at least one electronic health record data object.

For example, the computing entity may generate a member demographics data object for each piece of the demographic information from the at least one electronic health record data object, and may determine whether the healthcare graph data object comprises an attribute vertex that corresponds to each member demographics data object. If the healthcare graph data object comprises a corresponding attribute vertex, the computing entity generates an edge that connects the member vertex to the corresponding attribute vertex. If the healthcare graph data object does not comprise a corresponding attribute vertex, the computing entity generates a corresponding attribute vertex and an edge that connects the member vertex to the corresponding attribute vertex.

Continuing from the Alex example above, the computing entity may generate a first member demographics data object indicating “67 years old,” a second member demographics data object indicating “male,” and a third member demographics data object indicating “Houston, Tex.” The computing entity may determine whether the healthcare graph data object comprises a first attribute vertex corresponding to the first member demographics data object, a second attribute vertex corresponding to the second member demographics data object, and a third attribute vertex corresponding to the third member demographics data object. If so, the computing entity connects the first attribute vertex, the second attribute vertex, and the third attribute vertex to the member vertex corresponding to Alex. If not, the computing entity generates the first attribute vertex, the second attribute vertex, and/or the third attribute vertex and then connects them to the member vertex that corresponds to Alex.

Referring back to FIG. 5, subsequent to and/or in response to step/operation 503, the example method 500 proceeds to step/operation 507. At step/operation 507, a computing entity (such as the query prediction computing entity 105 described above in connection with FIG. 1 and FIG. 2) may include means (such as the processing element 205 of the query prediction computing entity 105 described above in connection with FIG. 2) to generate the member history data object based at least in part on the health history information from the at least one electronic health record data object.

For example, the computing entity may generate a member history data object for each piece of the health history information from the at least one electronic health record data object, and may determine whether the healthcare graph data object comprises an attribute vertex that corresponds to each member history data object. If the healthcare graph data object comprises a corresponding attribute vertex, the computing entity connects the member vertex to the corresponding attribute vertex. If the healthcare graph data object does not comprise a corresponding attribute vertex, the computing entity generates a corresponding attribute vertex and connects the member vertex to the corresponding attribute vertex.

Continuing from the Alex example above, the computing entity may generate a first member history data object indicating “Type II diabetes,” a second member history data object indicating “hypertension,” and a third member history data object indicating “total knee replacement.” The computing entity may determine whether the healthcare graph data object comprises a first attribute vertex corresponding to the first member history data object, a second attribute vertex corresponding to the second member history data object, and a third attribute vertex corresponding to the third member history data object. If so, the computing entity connects the first attribute vertex, the second attribute vertex, and the third attribute vertex to the member vertex corresponding to Alex. If not, the computing entity generates the first attribute vertex, the second attribute vertex, and/or the third attribute vertex and then connects them to the member vertex that corresponds to Alex.

Referring back to FIG. 5, subsequent to and/or in response to step/operation 505 and/or step/operation 507, the example method 500 proceeds to step/operation 509 and ends.

While the description above provides some examples of generating member data objects, it is noted that the scope of the present disclosure is not limited to the description above. For example, as described above, an example electronic health record data object may comprise symptom information associated with the member. In this example, the computing entity may generate member symptom data object(s) based at least in part on the symptom information from the example electronic health record data object.

As described above, there are technical challenges, deficiencies and problems associated with database systems, and various example embodiments of the present disclosure overcome such challenges. For example, referring now to FIG. 6A and FIG. 6B, an example method 600 of generating member supplemental data objects in accordance with embodiments of the present disclosure is illustrated.

For example, the example method 600 may generate a visit vertex in the healthcare graph data object that is connected to the member vertex in response to receiving an actual member visit indication from a computing entity associated with the member identifier, and generate one or more member supplemental data objects based at least in part on the one or more supplemental data inputs that are extracted from textual inputs generated based on audio inputs and/or visual inputs. As such, the example method 600 may, for example but not limited to, improve relevancy, applicability, and accuracy associated with the predicted data.

As shown in FIG. 6A, the example method 600 starts at step/operation 602. Subsequent to and/or in response to step/operation 602, the example method 600 proceeds to step/operation 604. At step/operation 604, a computing entity (such as the query prediction computing entity 105 described above in connection with FIG. 1 and FIG. 2) may include means (such as the processing element 205 of the query prediction computing entity 105 described above in connection with FIG. 2) to receive an actual member visit indication from a computing entity associated with the member identifier.

In some embodiments, the computing entity may receive the actual member visit indication from a client computing entity associated with the member identifier prior to generating the one or more attribute vertices.

As described above, an actual member visit indication may be provided by a member to the graph-based query prediction platform/system indicating that the member is currently in a visit to a provider. For example, the graph-based query prediction platform/system may include one or more software application(s) or app(s) that are installed on a computing entity associated with a member (e.g. a mobile phone). The member may provide the actual member visit indication to the graph-based query prediction platform/system through, for example but not limited to, a graphic user interface of the app(s) that is rendered on a display of the computing entity.

For example, a member Alex may conduct a visit to a provider. At the beginning of or during the visit, Alex may provide user input to a computing entity (e.g. his mobile smartphone), causing an app associated with the graph-based query prediction platform/system to be executed. In some embodiments, Alex may provide a user input through the graphic user interface (for example, clicking/selecting a bottom on the graphic user interface) to provide an actual member visit indication to the graph-based query prediction platform/system as so to notify the graph-based query prediction platform/system that he is currently conducting a visit to a provider.

Referring back to FIG. 6A, subsequent to and/or in response to step/operation 604, the example method 600 proceeds to step/operation 606. At step/operation 606, a computing entity (such as the query prediction computing entity 105 described above in connection with FIG. 1 and FIG. 2) may include means (such as the processing element 205 of the query prediction computing entity 105 described above in connection with FIG. 2) to generate a visit vertex in the healthcare graph data object that is connected to the member vertex.

In some embodiments, the computing entity may generate the visit vertex in the healthcare graph data object and connect the visit vertex to the member vertex in response to receiving the actual member visit indication described above in connection with step/operation 604. As described above, the visit vertex refers to a point and/or a node in an example healthcare graph data object that corresponds to, indicates, and illustrates a visit by a member to a provider, and may be associated with a visit identifier.

Continuing from the Alex example above, the computing entity may generate a visit vertex that corresponds to the visit as indicated by the actual member visit indication received at step/operation 604, and may connect the member vertex that corresponds to Alex to the visit vertex generated at step/operation 606.

Referring back to FIG. 6A, subsequent to and/or in response to step/operation 606, the example method 600 proceeds to block A, which connects FIG. 6A to FIG. 6B. Referring now to FIG. 6B, as shown, subsequent to and/or in response to step/operation 606, the example method 600 proceeds to step/operation 608. At step/operation 608, a computing entity (such as the query prediction computing entity 105 described above in connection with FIG. 1 and FIG. 2) may include means (such as the processing element 205 of the query prediction computing entity 105 described above in connection with FIG. 2) to receive, from the computing entity associated with the member identifier, one or more audio inputs or one or more visual inputs.

As described above, audio input may refer to user input to a computing entity that is in an audio form such as, but not limited to, member voices and/or conversations between a member and a provider as detected by a microphone of the computing entity, and/or the like. As described above, visual input may refer to user input to a computing entity that is in a visual form such as, but not limited to, one or more pictures associated with a member's visit that are captured through a camera of the computing entity during the member's visit to the provider.

Continuing from the Alex example above, through the app associated with the graph-based query prediction platform/system and installed on the computing entity, conversions between Alex and the provider may be captured by the microphone of the computing entity so as to provide audio input to the computing entity. Additionally, or alternatively, Alex may capture images of documents such as brochures received from the provider during the visit so as to provide visual inputs to the computing entity.

Referring back to FIG. 6B, subsequent to and/or in response to step/operation 608, the example method 600 proceeds to step/operation 610. At step/operation 610, a computing entity (such as the query prediction computing entity 105 described above in connection with FIG. 1 and FIG. 2) may include means (such as the processing element 205 of the query prediction computing entity 105 described above in connection with FIG. 2) to generate one or more textual inputs based at least in part on the one or more audio inputs or the one or more visual inputs.

In some embodiments, the computing entity may implement applicable software algorithms to convert the audio inputs and/or the visual inputs received at step/operation 608 into textual inputs. For example, the computing entity may implement speech-to-text algorithms to generate textual inputs based on the audio inputs received at step/operation 608. Additionally, or alternatively, the computing entity may implement optical character recognition (OCR) algorithms to generate textual inputs based on the visual inputs received at step/operation 608.

Continuing from the Alex example above, the computing entity may generate textual inputs based at least in part on the conversions between Alex and the provider that is captured by the microphone of the computing entity as audio inputs through speech-to-text algorithms. Additionally, or alternatively, the computing entity may generate textual inputs based at least in part on images of documents such as brochures captured by the camera of the computing entity as visual inputs through OCR algorithms.

Referring back to FIG. 6B, subsequent to and/or in response to step/operation 610, the example method 600 proceeds to step/operation 612. At step/operation 612, a computing entity (such as the query prediction computing entity 105 described above in connection with FIG. 1 and FIG. 2) may include means (such as the processing element 205 of the query prediction computing entity 105 described above in connection with FIG. 2) to extract, using at least one natural language processing model, one or more supplemental data inputs from the one or more textual inputs.

As described above, the at least one natural language processing model may process and analyze natural language data such as textual inputs generated at step/operation 610. In some embodiments, by using the at least one natural language processing model, the computing entity may extract data and/or information from the textual inputs that are associated with and/or related to demographic information, health history information, symptom information, and/or the like as described above, and designate such data and/or information as supplemental data inputs at step/operation 612.

Continuing from the Alex example above, the computing entity may extract demographic information (e.g. 67 years old) from the textual inputs generated based on the audio inputs. Additionally, or alternatively, the computing entity may extract health history information (e.g. Type II diabetes) from the textual inputs generated based on the visual inputs. The computing entity may designate these demographic information and health history information as supplemental data inputs as step/operation 612.

Referring back to FIG. 6B, subsequent to and/or in response to step/operation 612, the example method 600 proceeds to step/operation 614. At step/operation 614, a computing entity (such as the query prediction computing entity 105 described above in connection with FIG. 1 and FIG. 2) may include means (such as the processing element 205 of the query prediction computing entity 105 described above in connection with FIG. 2) to generate one or more member supplemental data objects based at least in part on the one or more supplemental data inputs.

In some embodiments, the computing entity may generate a member supplemental data object for each piece of information from supplemental data input that is extracted at step/operation 612.

In some embodiments, the computing entity determines whether the healthcare graph data object comprises an attribute vertex that corresponds to each member supplemental data object generated at step/operation 614. If not, the computing entity may generate an attribute vertex that corresponds to the member supplemental data object.

In some embodiments, the computing entity may generate one or more supplemental edges connecting one or more supplemental attribute vertices to the member vertex based at least in part on the one or more member supplemental data objects, similar to those described above in connection with at least step/operation 404 of FIG. 4.

In some embodiments, the one or more member data objects described above in connection with at least step/operation 404 of FIG. 4 comprises the one or more member supplemental data objects generated at step/operation 614 of FIG. 6B. As such, the example method 600 provides an example of dynamically updating a healthcare graph data object and dynamically generating predicted member query vertices during a member's visit to the provider. For example, as new member supplemental data objects are generated at step/operation 614 based at least in part on audio inputs and/or visual inputs capture during the visit, similar to the example method 400 describe above in connection with FIG. 4, the computing entity may generate one or more edges connecting one or more attribute vertices associated with the new member supplemental data objects to the member vertex in the healthcare graph data object based at least in part on the determining one or more historical member vertices from the healthcare graph data object, determining one or more historical member query vertices from the healthcare graph data object, generating one or more predicted member query vertices in the healthcare graph data object, and performing one or more prediction-based actions based at least in part on the one or more predicted member query vertices. Similarly, the computing entity may dynamically update the healthcare graph data object and dynamically generate predicted provider query vertices during a member's visit to the provider based on those examples described herein.

Continuing from the Alex example above, the graph-based query prediction platform/system may dynamically generate one or more predicted member query vertices based at least in part on information collected during Alex's visit to the provider, and may dynamically generate predicted member query indications on the graphic user interface based on the one or more predicted member query vertices.

Referring back to FIG. 6B, subsequent to and/or in response to step/operation 614, the example method 600 proceeds to step/operation 616 and ends.

While the description above provides an example of dynamically generating predicted member query vertices during the visit, it is noted that the scope of the present disclosure is not limited to the description above. In some examples, an example method may comprise one or more additional and/or alternative predicted member query vertices prior to a visit.

For example, while FIG. 6A to FIG. 6B illustrates that an actual member visit indication may trigger generating predicted member query vertices in accordance with various embodiments described herein, in some embodiments, an anticipatory member visit indication may additionally or alternatively trigger generating predicted member query vertices and/or the predicted member query vertices in accordance with various embodiments described herein. As described above, the example anticipatory member visit indication refers to an indication provided by a member that the member plans to conduct a visit to a provider in the future. In some embodiments, in response to receiving the anticipatory member visit indication, the computing entity may generate predicted provider query vertices in accordance with various examples described herein so as to allow the member to “preview” questions that may be asked by the provider during the visit, and/or may generate predicted member query vertices in accordance with various examples described herein.

### e. Exemplary Techniques for Calculating Resemblance Metrics

Referring now to FIG. 7, an example diagram illustrating a portion of an example healthcare graph data object 700 is provided.

As described above, an example healthcare graph data object may represent, organize, and/or store healthcare data and/or information in the form of vertices, and may, for example, utilize one or more edges connecting vertices to indicate, illustrate, and/or specify relationships between healthcare data and/or information.

In the example shown in FIG. 7, the example healthcare graph data object 700 comprises a plurality of member vertices (such as, but not limited to, member vertex M1, member vertex M2, member vertex M3, and member vertex M4). As described above, each of the plurality of member vertices corresponds to, indicates, and illustrates a member associated with the example graph-based query prediction platform/system.

In the example shown in FIG. 7, the example healthcare graph data object 700 also comprises a plurality of attribute vertices (such as, but not limited to, attribute vertex A1, attribute vertex A2, attribute vertex A3, and attribute vertex A4). As described above, each of the plurality of attribute vertices corresponds to, indicates, and illustrates data and/or information of one or more attributes associated with a member.

In some embodiments, an edge connecting a member vertex and an attribute vertex indicates that the member corresponding to the member vertex has or is associated with the attribute corresponding to the attribute vertex. In the example shown in FIG. 7, the member vertex M1 is connected to the attribute vertex A1 and the attribute vertex A2, which indicates that the member corresponding to the member vertex M1 has or is associated with the attributes corresponding to the attribute vertex A1 and the attribute vertex A2. The member vertex M2 is connected to the attribute vertex A1, the attribute vertex A2, and the attribute vertex A3, which indicates that the member corresponding to the member vertex M2 has or is associated with the attributes corresponding to the attribute vertex A1, the attribute vertex A2, and the attribute vertex A3. The member vertex M3 is connected to the attribute vertex A2, which indicates that the member corresponding to the member vertex M3 has or is associated with the attribute corresponding to the attribute vertex A2. The member vertex M4 is connected to the attribute vertex A4, which indicates that the member corresponding to the member vertex M4 has or is associated with the attribute corresponding to the attribute vertex A4.

As described above, an example healthcare graph data object may comprise a member subgraph and one or more historical member subgraphs. For example, the member subgraph comprises the member vertex and the one or more attribute vertices that are connected to the member vertex and associated with the member vertex. Each of the one or more historical member subgraphs comprises a historical member vertex of the one or more historical member vertices and at least one attribute vertex from an attribute vertex set that is connected to the historical member vertex and is associated with the historical member vertex.

As an example, a computing entity may retrieve the healthcare graph data object 700 to generate one or more predicted member query vertices for the member vertex M1. In this example, member vertices other than the member vertex M1 (such as member vertex M2, member vertex M3, and member vertex M4) are historical member vertices.

Continuing from the example above, the member subgraph associated with the member vertex M1 may include the member vertex M1 and the attribute vertex A1 and the attribute vertex A2. The historical member subgraph associated with the member vertex M2 may include the historical member vertex M2 and the attribute vertex A1, the attribute vertex A2, and the attribute vertex A3. The historical member subgraph associated with the member vertex M3 may include the historical member vertex M3 and the attribute vertex A2. The historical member subgraph associated with the member vertex M4 may include the historical member vertex M4 and the attribute vertex A4.

While the description above provides some examples of member subgraphs and historical member subgraphs, it is noted that the scope of the present disclosure is not limited to the description above. In some examples, example member subgraphs and example historical member subgraphs may comprise one or more additional and/or alternative vertices. For example, example member subgraphs and/or example historical member subgraphs may comprise one or more visit vertices that are connected to the member vertex and/or the historical member vertex, and/or may comprise one or more provider vertices that are connected to the member vertex and/or the historical member vertex through the one or more visit vertices.

In some embodiments, a computing entity may calculate resemblance metrics between a member vertex and one or more historical member vertices and/or calculate resemblance metrics between a member subgraph and one or more historical member subgraphs, details of which are described herein.

As described above, there are technical challenges, deficiencies and problems associated with database systems, and various example embodiments of the present disclosure overcome such challenges. For example, referring now to FIG. 8, an example method 800 of calculating resemblance metrics in accordance with embodiments of the present disclosure is illustrated.

For example, the example method 800 may calculate cosine similarity measures and determine resemblance metrics based at least in part on cosine similarity measures. As such, the example method 800 may, for example but not limited to, improve relevancy, applicability, and accuracy associated with the predicted data.

As shown in FIG. 8, the example method 800 starts at step/operation 802. Subsequent to and/or in response to step/operation 802, the example method 800 proceeds to step/operation 804. At step/operation 804, a computing entity (such as the query prediction computing entity 105 described above in connection with FIG. 1 and FIG. 2) may include means (such as the processing element 205 of the query prediction computing entity 105 described above in connection with FIG. 2) to calculate the one or more resemblance metrics based at least in part on the one or more attribute vertices and the attribute vertex set.

In some embodiments, the one or more attribute vertices are associated with the member vertex. In some embodiments, an attribute vertex set is associated with each of the one or more historical member vertices.

For example, referring back to the example described in connection with FIG. 7 above, the one or more attribute vertices that are associated with the member vertex M1 include attribute vertex A1 and attribute vertex A2. The attribute vertex set that is associated with the historical member vertex M2 includes the attribute vertex A1, the attribute vertex A2, and the attribute vertex A3.

In the example shown in FIG. 8, step/operation 804 may comprise one or more additional steps/operations.

In some embodiments, step/operation 804 may include step/operation 808. At step/operation 808, a computing entity (such as the query prediction computing entity 105 described above in connection with FIG. 1 and FIG. 2) may include means (such as the processing element 205 of the query prediction computing entity 105 described above in connection with FIG. 2) to calculate one or more cosine similarity measures between the member subgraph of the healthcare graph data object and each of the one or more historical member subgraphs of the healthcare graph data object.

As described above, cosine similarity measures refer to a type of similarity measure that is defined based on the cosine of the angle between the two or more vertices and/or two or more subgraphs.

For example, in connection with the example described in connection with FIG. 7 above, the computing entity may calculate a cosine similarity measure between the member subgraph comprising the member vertex M1, the attribute vertex A1 and the attribute vertex A2 and the historical member subgraph comprising historical member vertex M2, the attribute vertex A1, the attribute vertex A2, and the attribute vertex A3. Additionally, or alternatively, the computing entity may calculate a cosine similarity measure between the member subgraph comprising the member vertex M1, the attribute vertex A1 and the attribute vertex A2 and the historical member subgraph comprising historical member vertex M3 and the attribute vertex A2. Additionally, or alternatively, the computing entity may calculate a cosine similarity measure between the member subgraph comprising the member vertex M1, the attribute vertex A1 and the attribute vertex A2 and the historical member subgraph comprising historical member vertex M4 and the attribute vertex A4.

While the description above provides some examples of member subgraphs and historical member subgraphs for calculating cosine similarity measures, it is noted that the scope of the present disclosure is not limited to the description above. In some examples, example member subgraphs and example historical member subgraphs may comprise one or more additional and/or alternative vertices. For example, example member subgraphs and/or example historical member subgraphs may comprise one or more visit vertices that are connected to the member vertex and/or the historical member vertex, and/or may comprise one or more provider vertices that are connected to the member vertex and/or the historical member vertex through the one or more visit vertices. In some embodiments, the cosine similarity measures may be calculated based further on the visit vertices and/or provider vertices that are connected to the member vertices and/or the historical member vertices directly/indirectly.

While the description above provides an example of using cosine similarity measures, it is noted that the scope of the present disclosure is not limited to the description above. In some examples, an example method may utilize other similarity measures, such as, but not limited to, Jaccard, and/or the like.

Referring back to FIG. 8, in some embodiments, step/operation 804 may include step/operation 810. For example, subsequent to and/or in response to step/operation 808, the example method 800 proceeds to step/operation 810. At step/operation 810, a computing entity (such as the query prediction computing entity 105 described above in connection with FIG. 1 and FIG. 2) may include means (such as the processing element 205 of the query prediction computing entity 105 described above in connection with FIG. 2) to determine the one or more resemblance metrics based at least in part on the one or more cosine similarity measures.

For example, in some embodiments, the computing entity may determine the one or more cosine similarity measures as the one or more resemblance metrics associated with the historical member vertices, and may determine the one or more historical member vertices at least according to the cosine similarity measures, similar to those described in connection with at least step/operation 406 of FIG. 4.

For example, the computing entity may determine/select historical member vertices associated with cosine similarity measures satisfying a predetermined threshold. Additionally, or alternatively, the computing entity may rank the one or more historical member vertices according to their corresponding cosine similarity measures, and may determine/select a predetermined number of historical member vertices from the top of the ranking. As such, the computing entity may determine/select historical member vertices that are sufficiently similar to the member vertex based at least in part on the cosine similarity measures associated with their corresponding subgraphs.

Continuing from the example above in connection with FIG. 7, in some embodiments, the computing entity may determine the resemblance metrics associated with the historical member vertex M2, the historical member vertex M3, and the historical member vertex M4 based at least in part on their corresponding cosine similar measures calculated at step/operation 808. In some embodiments, the cosine similar measures may be used to determine/select one or more historical member vertices from the historical member vertex M2, the historical member vertex M3, and the historical member vertex M4 that are sufficiently similar to the member vertex M1.

Referring back to FIG. 8, subsequent to and/or in response to step/operation 804, the example method 800 proceeds to step/operation 806 and ends.

As described above, there are technical challenges, deficiencies and problems associated with database systems, and various example embodiments of the present disclosure overcome such challenges. For example, referring now to FIG. 9, an example method 900 of calculating resemblance metrics in accordance with embodiments of the present disclosure is illustrated.

For example, the example method 900 may calculate the one or more resemblance metrics based at least in part on at least one graph-based machine learning model. As such, the example method 900 may, for example but not limited to, improve relevancy, applicability, and accuracy associated with the predicted data.

As shown in FIG. 9, the example method 900 starts at step/operation 901. Subsequent to and/or in response to step/operation 901, the example method 900 proceeds to step/operation 903. At step/operation 903, a computing entity (such as the query prediction computing entity 105 described above in connection with FIG. 1 and FIG. 2) may include means (such as the processing element 205 of the query prediction computing entity 105 described above in connection with FIG. 2) to calculate the one or more resemblance metrics based at least in part on the at least one graph-based machine learning model.

As described above, the at least one graph-based machine learning model may be trained to process and/or analyze one or more healthcare graph data objects and generate metrics based on the one or more healthcare graph data objects. Examples of graph-based machine learning models may include, but are not limited to, artificial neural networks, graph-neural networks, graph neural networks, and/or the like.

In the example shown in FIG. 9, step/operation 903 may comprise one or more additional steps/operations.

In some embodiments, step/operation 903 may include step/operation 907. At step/operation 907, a computing entity (such as the query prediction computing entity 105 described above in connection with FIG. 1 and FIG. 2) may include means (such as the processing element 205 of the query prediction computing entity 105 described above in connection with FIG. 2) to train the at least one graph-based machine learning model.

In some embodiments, the computing entity may train the at least one graph-based machine learning model based at least in part on one or more training healthcare graph data objects. As described, each of the one or more training healthcare graph data objects comprises a plurality of training member subgraphs.

In some embodiments, one or more training healthcare graph data objects are associated with one or more training resemblance metrics. For example, each of the one or more training resemblance metrics indicates a known resemblance metrics associated with two of the plurality of training member subgraphs.

In some embodiments, to train a graph-based machine learning model, the computing entity provides a training healthcare graph data object to the graph-based machine learning model, and the graph-based machine learning model generates a resemblance metrics associated with two of training member subgraphs of the training healthcare graph data object. In some embodiments, the resemblance metrics generated by the graph-based machine learning model is compared with the training resemblance metrics associated with the two of training member subgraphs, and the graph-based machine learning model may adjust its paraments so that the resemblance metrics is close to or matches the training resemblance metrics.

Referring back to FIG. 9, in some embodiments, step/operation 903 may include step/operation 909. For example, subsequent to and/or in response to step/operation 907, the example method 900 proceeds to step/operation 909. At step/operation 909, a computing entity (such as the query prediction computing entity 105 described above in connection with FIG. 1 and FIG. 2) may include means (such as the processing element 205 of the query prediction computing entity 105 described above in connection with FIG. 2) to input the healthcare graph data object to the at least one graph-based machine learning model.

In some embodiments, the computing entity may input the healthcare graph data object to the at least one graph-based machine learning model subsequent to training the at least one graph-based machine learning model to generate resemblance metrics described in connection with step/operation 907.

In some embodiments, step/operation 903 may include step/operation 911. For example, subsequent to and/or in response to step/operation 909, the example method 900 proceeds to step/operation 911. At step/operation 911, a computing entity (such as the query prediction computing entity 105 described above in connection with FIG. 1 and FIG. 2) may include means (such as the processing element 205 of the query prediction computing entity 105 described above in connection with FIG. 2) to receive the one or more resemblance metrics from the at least one graph-based machine learning model.

In some embodiments, the computing entity may receive the one or more resemblance metrics from the at least one graph-based machine learning model. In some embodiments, each of the one or more resemblance metrics indicates a similarity measure between one of the one or more historical member vertices and the member vertex. In some embodiments, the computing entity may determine the one or more historical member vertices at least according to the one or more resemblance metrics, similar to those described in connection with at least step/operation 406 of FIG. 4.

For example, the computing entity may determine/select historical member vertices associated with resemblance metrics satisfying a predetermined threshold. Additionally, or alternatively, the computing entity may rank the one or more historical member vertices according to their corresponding resemblance metrics, and may determine/select a predetermined number of historical member vertices from the top of the ranking. As such, the computing entity may determine/select historical member vertices that are sufficiently similar to the member vertex based at least in part on their corresponding resemblance metrics.

Referring back to FIG. 9, subsequent to and/or in response to step/operation 903, the example method 900 proceeds to step/operation 905 and ends.

### f. Exemplary Techniques for Calculating Prioritization Metrics

As described above, there are technical challenges, deficiencies and problems associated with database systems, and various example embodiments of the present disclosure overcome such challenges. For example, referring now to FIG. 10, an example method 1000 of determining prioritization metrics in accordance with embodiments of the present disclosure is illustrated.

For example, the example method 1000 may determine a member-query edge weight associated with the one or more edges for the historical member query vertex. As such, the example method 1000 may, for example but not limited to, improve relevancy, applicability, and accuracy associated with the predicted data.

As shown in FIG. 10, the example method 1000 starts at step/operation 1002. Subsequent to and/or in response to step/operation 1002, the example method 1000 proceeds to step/operation 1004. At step/operation 1004, a computing entity (such as the query prediction computing entity 105 described above in connection with FIG. 1 and FIG. 2) may include means (such as the processing element 205 of the query prediction computing entity 105 described above in connection with FIG. 2) to determine a member-query edge weight for the historical member query vertex associated with one or more edges.

In some embodiments, the historical member query vertex is connected to the historical member vertex through the one or more edges. As described above, a member-query edge weight may indicate a priority of a historical member query vertex relative to a historical member vertex.

For example, a historical member vertex may be connected to a plurality of historical member query vertices through a plurality of member-query edges, and the computing entity may calculate a member-query edge weight for each of the plurality of member-query edges. In this example, the member-query edge weight indicates a priority of a historical member query vertex among other historical member query vertices that are connected to the historical member vertex.

In some embodiments, the computing entity may determine the member-query edge weight based at least in part on the healthcare outcome measure, details of which are described in connection with at least FIG. 11. In some embodiments, the computing entity may determine the member-query edge weight based at least in part on the healthcare cost measure, details of which are described in connection with at least FIG. 12.

Referring back to FIG. 10, subsequent to and/or in response to step/operation 1004, the example method 1000 proceeds to step/operation 1006. At step/operation 1006, a computing entity (such as the query prediction computing entity 105 described above in connection with FIG. 1 and FIG. 2) may include means (such as the processing element 205 of the query prediction computing entity 105 described above in connection with FIG. 2) to determine, for the historical member query vertex, a prioritization metrics of the one or more prioritization metrics based at least in part on the member-query edge weight.

In some embodiments, the computing entity may set the prioritization metrics associated with a historical member query vertex based on the member-query edge weight associated with the historical member query vertex. For example, a historical member query vertex associated with a higher member-query edge weight is assigned to a higher prioritization metrics.

In some embodiments, the computing entity may determine/select one or more historical member query vertices based at least in part on the prioritization metrics, similar to those described above in connection with at least step/operation 408 of FIG. 4.

Referring back to FIG. 10, subsequent to and/or in response to step/operation 1006, the example method 1000 proceeds to step/operation 1008 and ends.

As described above, there are technical challenges, deficiencies and problems associated with database systems, and various example embodiments of the present disclosure overcome such challenges. For example, referring now to FIG. 11, an example method 1100 of calculating member-query edge weights in accordance with embodiments of the present disclosure is illustrated.

For example, the example method 1100 may calculate member-query edge weights based at least in part on the healthcare outcome measures. As such, the example method 1100 may, for example but not limited to, improve relevancy, applicability, and accuracy associated with the predicted data.

As shown in FIG. 11, the example method 1100 starts at Block A, which continues from step/operation 1006 of FIG. 10 described above. Subsequent to and/or in response to Block A, the example method 1100 proceeds to step/operation 1101. At step/operation 1101, a computing entity (such as the query prediction computing entity 105 described above in connection with FIG. 1 and FIG. 2) may include means (such as the processing element 205 of the query prediction computing entity 105 described above in connection with FIG. 2) to determine a healthcare outcome measure associated with the historical member vertex.

As described above, a healthcare outcome measure may refer to a measure or a parameter that is associated with a member identifier and/or a visit identifier, and may quantitatively or qualitatively indicate, illustrate, and/or specify the quality, effectiveness and/or successfulness of a member's visit to a provider.

In some embodiments, healthcare outcome measures may be stored in a database that is internal to a graph-based query prediction platform/system in accordance with various examples described herein. In some embodiments, healthcare outcome measures may be stored in a database that is external to a graph-based query prediction platform/system in accordance with various examples described herein.

For example, a healthcare outcome measure may be associated with a member Alex, which may quantitatively or qualitatively indicate, illustrate, and/or specify the quality, effectiveness and/or successfulness of Alex's visit to the provider. For example, the healthcare outcome measure may quantitatively indicate how quickly Alex recovered from a disease after Alex's visit (e.g. a recovery time). Additionally, or alternatively, the healthcare outcome measure may indicate other aspects associated with the quality, effectiveness and/or successfulness of Alex's visit to the provider.

Referring back to FIG. 11, subsequent to and/or in response to step/operation 1101, the example method 1100 proceeds to step/operation 1103. At step/operation 1103, a computing entity (such as the query prediction computing entity 105 described above in connection with FIG. 1 and FIG. 2) may include means (such as the processing element 205 of the query prediction computing entity 105 described above in connection with FIG. 2) to calculate the member-query edge weight based at least in part on the healthcare outcome measure.

In some embodiments, the computing entity may factor in the healthcare outcome measure when calculating the member-query edge weight. As described above, the member-query edge weight may be associated with one or more edges connecting a historical member vertex and a historical member query vertex. In some embodiments, the historical member vertex is associated with a member identifier, and the computing entity may factor in a healthcare outcome measure associated with the member identifier when calculating the member-query edge weight. In some embodiments, the historical member query vertex is associated with a visit identifier (e.g. the question associated with the historical member query vertex is asked during a visit associated with the visit identifier), and the computing entity may factor in a healthcare outcome measure associated with the visit identifier when calculating the member-query edge weight.

In some embodiments, the computing entity may set the healthcare outcome measure such that the higher the healthcare outcome measure, the higher the member-query edge weight. In some embodiments, a change in the healthcare outcome measure is proportional to a change in the member-query edge weight. In some embodiments, the member-query edge weight is the same as the healthcare outcome measure. As such, various embodiments of the present disclosure prioritizes questions asked by other members who had better outcomes.

Referring back to FIG. 11, subsequent to and/or in response to step/operation 1103, the example method 1100 proceeds to Block B, which returns after step/operation 1006 of FIG. 10 and ends.

As described above, there are technical challenges, deficiencies and problems associated with database systems, and various example embodiments of the present disclosure overcome such challenges. For example, referring now to FIG. 12, an example method 1200 of calculating member-query edge weights in accordance with embodiments of the present disclosure is illustrated.

For example, the example method 1200 may calculate member-query edge weights based at least in part on the healthcare cost measures. As such, the example method 1200 may, for example but not limited to, improve relevancy, applicability, and accuracy associated with the predicted data.

As shown in FIG. 12, the example method 1200 starts at Block A, which continues from step/operation 1006 of FIG. 10 described above. Subsequent to and/or in response to Block A, the example method 1200 proceeds to step/operation 1202. At step/operation 1202, a computing entity (such as the query prediction computing entity 105 described above in connection with FIG. 1 and FIG. 2) may include means (such as the processing element 205 of the query prediction computing entity 105 described above in connection with FIG. 2) to determine a healthcare cost measure associated with the historical member vertex.

As described above, a healthcare cost measure may refer to a measure or a parameter that is associated with a member identifier and/or a visit identifier, and may quantitatively or qualitatively indicate, illustrate, and/or specify a healthcare cost associated with the member and/or the visit.

In some embodiments, healthcare cost measures may be stored in a database that is internal to a graph-based query prediction platform/system in accordance with various examples described herein. In some embodiments, healthcare cost measures may be stored in a database that is external to a graph-based query prediction platform/system in accordance with various examples described herein.

For example, a healthcare cost measure may be associated with a member Alex, which may quantitatively or qualitatively indicate, illustrate, and/or specify a healthcare cost associated with Alex and/or Alex's visit to a provider. For example, the healthcare outcome measure may quantitatively indicate the cost of medication prescribed by the provider for Alex during the visit. Additionally, or alternatively, the healthcare outcome measure may indicate other aspects associated with the cost of Alex's visit to the provider.

Referring back to FIG. 12, subsequent to and/or in response to step/operation 1202, the example method 1200 proceeds to step/operation 1204. At step/operation 1204, a computing entity (such as the query prediction computing entity 105 described above in connection with FIG. 1 and FIG. 2) may include means (such as the processing element 205 of the query prediction computing entity 105 described above in connection with FIG. 2) to calculate the member-query edge weight based at least in part on the healthcare cost measure.

In some embodiments, the computing entity may factor in the healthcare cost measure when calculating the member-query edge weight. As described above, the member-query edge weight may be associated with one or more edges connecting a historical member vertex and a historical member query vertex. In some embodiments, the historical member vertex is associated with a member identifier, and the computing entity may factor in a healthcare cost measure associated with the member identifier when calculating the member-query edge weight. In some embodiments, the historical member query vertex is associated with a visit identifier (e.g. the question associated with the historical member query vertex is asked during a visit associated with the visit identifier), and the computing entity may factor in a healthcare cost measure associated with the visit identifier when calculating the member-query edge weight.

In some embodiments, the computing entity may set the healthcare cost measure such that the higher the healthcare cost measure higher, the lower the member-query edge weight. In some embodiments, a change in the healthcare cost measure is proportional to a change in the member-query edge weight. In some embodiments, the member-query edge weight is one divided by the healthcare outcome measure. As such, various embodiments of the present disclosure prioritizes questions asked by other members who had lower costs.

Referring back to FIG. 12, subsequent to and/or in response to step/operation 1204, the example method 1200 proceeds to Block B, which returns after step/operation 1006 of FIG. 10 and ends.

While the description above in connection with FIG. 11 and FIG. 12 illustrate examples of calculating the member-query edge weight based on the healthcare outcome measure or the healthcare cost measure, it is noted that the scope of the present disclosure is not limited to the description above. In some examples, an example method may calculate the member-query edge weight through other ways. For example, an example method may calculate the member-query edge weight based on a ratio of the healthcare outcome measure as divided by the healthcare cost measure.

Referring now to FIG. 13, an example diagram illustrating a portion of an example healthcare graph data object 1300 is provided.

As described above, an example healthcare graph data object may comprise a historical visit vertex that corresponds to, indicates, and illustrates a visit by a member to a provider. In some embodiments, the healthcare graph data object 1300 comprises a plurality of historical visit vertices, such as, but not limited to, historical visit vertex V1, historical visit vertex V2, historical visit vertex V3, and historical visit vertex V4.

In some embodiments, a historical visit vertex of the plurality of historical visit vertices is connected to a historical member vertex and a historical member query vertex. Referring now to the example shown in FIG. 13, the historical visit vertex V1 is connected to the member vertex M3, the provider vertex P2, the historical member query vertex HMQ1, and the historical member query vertex HMQ2, which indicates that the member corresponds to the member vertex M3 asked the provider corresponding to the provider vertex P2 the questions represented by the historical member query vertex HMQ1 and the historical member query vertex HMQ2 during the visit represented by the historical visit vertex V1.

As another example, the historical visit vertex V2 is connected to the member vertex M2, the provider vertex P1, the historical member query vertex HMQ2, and the historical member query vertex HMQ3, which indicates that the member corresponds to the member vertex M2 asked the provider corresponding to the provider vertex P1 the questions represented by the historical member query vertex HMQ2 and the historical member query vertex HMQ3 during the visit represented by the historical visit vertex V2.

As another example, the historical visit vertex V3 is connected to the member vertex M4, the provider vertex P1, and the historical member query vertex HMQ3, which indicates that the member corresponds to the member vertex M4 asked the provider corresponding to the provider vertex P1 the question represented by the historical member query vertex HMQ3 during the visit represented by the historical visit vertex V3.

As another example, the historical visit vertex V4 is connected to the member vertex M4, the provider vertex P1, and the historical member query vertex HMQ4, which indicates that the member corresponds to the member vertex M4 asked the provider corresponding to the provider vertex P1 the question represented by the historical member query vertex HMQ4 during the visit represented by the historical visit vertex V4.

In some embodiments, the one or more edges that connect a historical member vertex to a historical member query vertex comprise a member-visit edge connecting the historical member vertex to the historical visit vertex and a visit-query edge connecting the historical visit vertex to the historical member query vertex. In some embodiments, the member-query edge weight is a combination of a member-visit edge weight associated with the member-visit edge and a visit-query edge weight associated with the visit-query edge.

As an example, as shown in FIG. 13, the one or more edges that connect a historical member vertex M3 to a historical member query vertex HMQ1 comprises a member-visit edge connecting the historical member vertex M1 to the historical visit vertex V1 and a visit-query edge connecting the historical visit vertex V1 to the historical member query vertex HMQ1. In some embodiments, the member-query edge weight is a combination of a member-visit edge weight associated with the member-visit edge connecting the historical member vertex M1 to the historical visit vertex V1 and a visit-query edge weight associated with the visit-query edge connecting the historical visit vertex V1 to the historical member query vertex HMQ1.

In the example shown in FIG. 13, predicted member query vertices (such as, but not limited to, PMQ1, PMQ2, PMQ3, and PMQ4) may be generated in accordance with various example methods of the present disclosure and may be connected to a corresponding member vertex. For example, predicted member query vertices PMQ1 and PMQ2 are generated for and connected to the member vertex M1, which indicates that the graph-based query prediction platform/system recommends the member represented by the member vertex M1 to ask questions represented by the predicted member query vertices PMQ1 and PMQ2 during the member's visit to a provider. As another example, predicted member query vertices PMQ3 and PMQ4 are generated for and connected to the member vertex M3, which indicates that the graph-based query prediction platform/system recommends the member represented by the member vertex M3 to ask questions represented by the predicted member query vertices PMQ3 and PMQ4 during the member's visit to a provider.

In the example shown in FIG. 13, predicted provider query vertices (such as, but not limited to, PPQ1) may be generated in accordance with various example methods of the present disclosure and may be connected to a corresponding member vertex and a corresponding provider vertex. For example, the predicted provider query vertex PPQ1 is connected to the member vertex M1 and the provider vertex P1, which indicates that the graph-based query prediction platform/system recommends the provider corresponding to the provider vertex P1 to ask the member corresponding to the member vertex M1 the question represented by the predicted provider query vertex PPQ1 during the member's visit to the provider.

### g. Exemplary Interactive User Interface

As described above, there are technical challenges, deficiencies and problems associated with database systems, and various example embodiments of the present disclosure overcome such challenges. For example, referring now to FIG. 14A and FIG. 14B, an example method 1400 of performing prediction-based actions based at least in part on predicted member query vertices in accordance with embodiments of the present disclosure is illustrated.

For example, the example method 1400 may receive a user input associated with a predicted member query indication and increase/decrease a member-query edge weight of a member-query edge. As such, the example method 1400 may, for example but not limited to, improve relevancy, applicability, and accuracy associated with the predicted data.

As shown in FIG. 14A, the example method 1400 starts at step/operation 1402. Subsequent to and/or in response to step/operation 1402, the example method 1400 proceeds to step/operation 1404. At step/operation 1404, a computing entity (such as the query prediction computing entity 105 described above in connection with FIG. 1 and FIG. 2) may include means (such as the processing element 205 of the query prediction computing entity 105 described above in connection with FIG. 2) to render one or more predicted member query indications based at least in part on the one or more predicted member query vertices.

In some embodiments, the computing entity may render the one or more predicted member query indications on a graphic user interface displayed on a client computing entity based at least in part on the one or more predicted member query vertices.

For example, the computing entity may render a predicted member query indication on the graphic user interface for each predicted member query vertex generated by the graph-based query prediction platform/system.

As described above, an example predicted member query vertex may include data and/or information associated with one or more question(s) that are determined/generated by an example graph-based query prediction platform/system and/or are recommended to a member for the member to ask a provider during the member's visit to the provider. As such, an example predicted member query indication on the graphic user interface may display the one or more question(s) that are determined/generated by an example graph-based query prediction platform/system.

Referring back to FIG. 14A, subsequent to and/or in response to step/operation 1404, the example method 1400 proceeds to step/operation 1406. At step/operation 1406, a computing entity (such as the query prediction computing entity 105 described above in connection with FIG. 1 and FIG. 2) may include means (such as the processing element 205 of the query prediction computing entity 105 described above in connection with FIG. 2) to receive a user input associated with a predicted member query indication that corresponds to a predicted member query vertex.

In some embodiments, the computing entity may receive the user input subsequent to rendering the one or more predicted member query indications. In some embodiments, the user input may be associated with a predicted member query indication of the one or more predicted member query indications. As described above, the one or more predicted member query indications may be generated based on and correspond to one or more predicted member query vertices. As such, the user input associated with the predicted member query indication also corresponds to the predicted member query vertex of the one or more predicted member query vertices.

Referring back to FIG. 14A, subsequent to and/or in response to step/operation 1406, the example method 1400 proceeds to step/operation 1408. At step/operation 1408, a computing entity (such as the query prediction computing entity 105 described above in connection with FIG. 1 and FIG. 2) may include means (such as the processing element 205 of the query prediction computing entity 105 described above in connection with FIG. 2) to determine a historical member query vertex that corresponds to the predicted member query vertex.

As described above in connection with step/operation 1406 of FIG. 14A, the user input associated with the predicted member query indication also corresponds to a predicted member query vertex. As described in connection with the at least step/operation 410 of FIG. 4, the computing entity may generate a predicted member query vertex for a member vertex based on a historical member query vertex that is associated with a historical member vertex similar to the member vertex. As such, the historical member query vertex corresponds to the predicted member query vertex.

For example, at step/operation 1406, the computing entity may receive a user input associated with a predicted member query indication that corresponds to a predicted member query vertex PMQ1, and the predicted member query vertex PMQ1 may be generated based on the historical member query vertex HMQ1. In this example, the historical member query vertex HMQ1 is associated with the user input.

Referring back to FIG. 14A, subsequent to and/or in response to step/operation 1408, the example method 1400 proceeds to Block A, which connects FIG. 14A to FIG. 14B. Referring now to FIG. 14B, subsequent to and/or in response to step/operation 1408, the example method 1400 proceeds to step/operation 1410. At step/operation 1410, a computing entity (such as the query prediction computing entity 105 described above in connection with FIG. 1 and FIG. 2) may include means (such as the processing element 205 of the query prediction computing entity 105 described above in connection with FIG. 2) to determine whether the user input indicates a user interest or a user disinterest of the predicted member query indication.

As described above, a user input may provide user feedback on questions generated by the graph-based query prediction platform/system. For example, a user input indicating a user disinterest of a predicted member query indication that corresponds to a question indicates that the member does not deem the question as appropriate or suitable for the member to ask during the visit to the provider. A user input indicating a user interest of a predicted member query indication that corresponds to a question indicates that the member deems or considers the question as appropriate or suitable for the member to ask during the visit to the provider.

If, at step/operation 1410, the computing entity determines that the user input indicates a user selection of the predicted member query indication, the example method 1400 proceeds to step/operation 1412. At step/operation 1412, a computing entity (such as the query prediction computing entity 105 described above in connection with FIG. 1 and FIG. 2) may include means (such as the processing element 205 of the query prediction computing entity 105 described above in connection with FIG. 2) to increase a member-query edge weight of a member-query edge.

In some embodiments, the computing entity may, in response to determining that the user input indicates the user interest, increase the member-query edge weight of the member-query edge that is associated with the historical member query vertex determined at step/operation 1408.

For example, at step/operation 1406, the computing entity may receive a user input indicating a user interest associated with a predicted member query indication that corresponds to a predicted member query vertex PMQ1, and the predicted member query vertex PMQ1 may be generated based on the historical member query vertex HMQ1 associated with the historical member vertex M1. In this example, the user input indicates that the question associated with the historical member query vertex HMQ1 is appropriate or suitable for the member to ask during the visit to provider, and the computing entity may increase the member-query edge weight associated with one or more edges connecting the historical member vertex M1 and the historical member query vertex HMQ1.

In some embodiments, by increasing the member-query edge weight, the prioritization metrics associated with the historical member query vertex is also increased, and the computing entity may prioritize predicted member query vertex generated based on the historical member query vertex over other predicted member query vertices in accordance with various example methods described herein.

Referring back to FIG. 14B, if, at step/operation 1410, the computing entity determines that the user input indicates a user disinterest of the predicted member query indication, the example method 1400 proceeds to step/operation 1414. At step/operation 1414, a computing entity (such as the query prediction computing entity 105 described above in connection with FIG. 1 and FIG. 2) may include means (such as the processing element 205 of the query prediction computing entity 105 described above in connection with FIG. 2) to decrease a member-query edge weight of a member-query edge.

In some embodiments, the computing entity may, in response to determining that the user input indicates the user disinterest, decrease the member-query edge weight of the member-query edge that is associated with the historical member query vertex.

For example, at step/operation 1406, the computing entity may receive a user input indicating a user disinterest associated with a predicted member query indication that corresponds to a predicted member query vertex PMQ1, and the predicted member query vertex PMQ1 may be generated based on the historical member query vertex HMQ1 associated with the historical member vertex M1. In this example, the user input indicates that the question associated with the historical member query vertex HMQ1 is not appropriate or suitable for the member to ask during the visit to provider, and the computing entity may decrease the member-query edge weight associated with one or more edges connecting the historical member vertex M1 and the historical member query vertex HMQ1.

In some embodiments, by decreasing the member-query edge weight, the prioritization metrics associated with the historical member query vertex is also decreased, and the computing entity may prioritize other predicted member query vertices over the predicted member query vertex generated based on the historical member query vertex based on various example methods described herein.

Referring back to FIG. 14B, subsequent to and/or in response to step/operation 1412 and/or step/operation 1414, the example method 1400 proceeds to step/operation 1416 and ends.

Referring now to FIG. 15, FIG. 16, FIG. 17, FIG. 18 and FIG. 19, example graphic user interfaces in accordance with various embodiments of the present disclosure are illustrated.

Referring now to FIG. 15 and FIG. 16, example graphic user interfaces 1500 and 1600 are illustrated, respectively. In particular, the example graphic user interface 1500 comprises a member information indication section 1501 and a predicted member query indication section 1503. The member information indication section 1501 may comprise renderings of member data objects such as, but not limited to, a member demographics data object, a member history data object, or a member symptom data object. The predicted member query indication section 1503 may comprise predicted member query indications that correspond to predicted member query vertices generated based on the member data objects rendered in the member information indication section 1501. Similarly, the example graphic user interface 1600 comprises a member information indication section 1602 and a predicted member query indication section 1604. The member information indication section 1602 may comprise renderings of member data objects such as, but not limited to, a member demographics data object, a member history data object, or a member symptom data object. The predicted member query indication section 1604 may comprise predicted member query indications that correspond to predicted member query vertices generated based on the member data objects rendered in the member information indication section 1602.

A comparison between the example graphic user interface 1500 and the example graphic user interface 1600 demonstrates that example embodiments generate personalized questions that can be recommended for a member to ask during the member's visit to a provider. For example, while the users associated with the example graphic user interface 1500 and the example graphic user interface 1600 both have symptoms such as pain in the leg and limping, they have different demographic information (for example, a 67-year-old male living in Houston shown in the example graphic user interface 1500 compared to a 24-year-old female living in California who is vegan shown in the example graphic user interface 1600) and have different health history information (for example, diabetes type II, hypertension, and total knee replacement shown in the example graphic user interface 1500 compared to none shown in the example graphic user interface 1600). Because of their different demographic information and/or different health history information, example embodiments of the present disclosure may generate different predicted member query vertices indicating different recommended questions, and/or may arrange the order of the predicted member query vertex indications corresponding to the different predicted member query vertices differently.

Referring now to FIG. 17 and FIG. 18, example graphic user interfaces 1700 and 1800 are illustrated, respectively. In particular, the example graphic user interface 1700 comprises a member information indication section 1701 and a predicted member query indication section 1703. The member information indication section 1701 may comprise renderings of member data objects such as, but not limited to, a member demographics data object, a member history data object, or a member symptom data object. The predicted member query indication section 1703 may comprise predicted member query indications that correspond to predicted member query vertices generated based on the member data objects rendered in the member information indication section 1701. Similarly, the example graphic user interface 1800 comprises a member information indication section 1802 and a predicted member query indication section 1804. The predicted member query indication section 1804 may comprise renderings of member data objects such as, but not limited to, a member demographics data object, a member history data object, or a member symptom data object. The predicted member query indication section 1804 may comprise predicted member query indications that correspond to predicted member query vertices generated based on the member data objects rendered in the member information indication section 1802.

In some embodiments, the example graphic user interface 1700 and the graphic user interface 1800 illustrate a dynamically changing graphic user interface during a member's visit to the provider. For example, the example graphic user interface 1700 may be a graphic user interface that is rendered one minute into the visit, and the example graphic user interface 1800 may be a graphic user interface that is rendered six minutes into the visit.

As shown in FIG. 18, the example graphic user interface 1800 may be dynamically updated to include, for example, a checkmark indication 1806A and a checkmark indication 1806B each next to a predicted member query indication corresponding to a question that the member has asked during the visit. For example, the computing entity may determine what questions that the member has asked based on the audio inputs described herein.

Additionally, or alternatively, the example graphic user interface 1800 may be dynamically updated to include new recommended questions and/or new rankings of recommended questions based at audio inputs and/or visual inputs received during the visit and in accordance with various examples described herein, including but not limited to, those described in connection with at least FIG. 4, FIG. 6A, and FIG. 6B above.

Referring now to FIG. 19, an example graphic user interface 1900 is illustrated. In particular, the example graphic user interface 1900 comprises a predicted provider query indication section 1901. The predicted provider query indication section 1901 may comprise predicted provider query indications that correspond to predicted provider query vertices generated in accordance with various examples described herein.

### V. CONCLUSION

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. An apparatus comprising at least one processor and at least one non-transitory memory comprising a computer program code, the at least one non-transitory memory and the computer program code configured to, with the at least one processor, cause the apparatus to:
generate one or more edges connecting one or more attribute vertices to a member vertex in a healthcare graph data object based at least in part on one or more member data objects comprising at least one of a member demographics data object, a member history data object, or a member symptom data object;
determine, using at least one graph-based machine learning model and based at least in part on the one or more attribute vertices, one or more historical member vertices from the healthcare graph data object at least according to one or more resemblance metrics associated with the one or more historical member vertices and relative to the member vertex;
determine, using the at least one graph-based machine learning model and based at least in part on the one or more historical member vertices, one or more historical member query vertices from the healthcare graph data object at least according to one or more prioritization metrics associated with the one or more historical member query vertices;
generate, based at least in part on the one or more historical member query vertices, one or more predicted member query vertices in the healthcare graph data object; and
perform one or more prediction-based actions based at least in part on the one or more predicted member query vertices.

2. The apparatus of claim 1, wherein the member vertex is associated with a member identifier, wherein the at least one non-transitory memory and the computer program code are configured to, with the at least one processor, cause the apparatus to:
retrieve at least one electronic health record data object associated with the member identifier, wherein the at least one electronic health record data object comprises demographic information and health history information associated with the member identifier;
generate the member demographics data object based at least in part on the demographic information from the at least one electronic health record data object; and
generate the member history data object based at least in part on the health history information from the at least one electronic health record data object.

3. The apparatus of claim 1, wherein the member vertex is associated with a member identifier, wherein the at least one non-transitory memory and the computer program code are configured to, with the at least one processor, cause the apparatus to:
prior to generating the one or more attribute vertices, receive an actual member visit indication from a client computing entity associated with the member identifier; and
in response to receiving the actual member visit indication, generate a visit vertex in the healthcare graph data object that is connected to the member vertex.

4. The apparatus of claim 3, wherein the at least one non-transitory memory and the computer program code are configured to, with the at least one processor, cause the apparatus to:
receive, from the client computing entity associated with the member identifier, one or more audio inputs or one or more visual inputs;
generate one or more textual inputs based at least in part on the one or more audio inputs or the one or more visual inputs;
extract, using at least one natural language processing model, one or more supplemental data inputs from the one or more textual inputs; and
generate one or more member supplemental data objects based at least in part on the one or more supplemental data inputs.

5. The apparatus of claim 4, wherein the one or more member data objects comprise the one or more member supplemental data objects, wherein the at least one non-transitory memory and the computer program code are configured to, with the at least one processor, cause the apparatus to generate one or more supplemental edges connecting one or more supplemental attribute vertices to the member vertex based at least in part on the one or more member supplemental data objects.

6. The apparatus of claim 1, wherein the at least one non-transitory memory and the computer program code are configured to, with the at least one processor, cause the apparatus to calculate the one or more resemblance metrics based at least in part on the one or more attribute vertices associated with the member vertex and an attribute vertex set associated with the one or more historical member vertices.

7. The apparatus of claim 6, wherein the healthcare graph data object comprises a member subgraph and one or more historical member subgraphs, wherein the member subgraph comprises the member vertex and the one or more attribute vertices that are connected to the member vertex and associated with the member vertex, wherein each of the one or more historical member subgraphs comprises a historical member vertex of the one or more historical member vertices and at least one attribute vertex from the attribute vertex set that is connected to the historical member vertex and is associated with the historical member vertex.

8. The apparatus of claim 7, wherein, when calculating the one or more resemblance metrics, the at least one non-transitory memory and the computer program code are configured to, with the at least one processor, cause the apparatus to:
calculate one or more cosine similarity measures between the member subgraph of the healthcare graph data object and each of the one or more historical member subgraphs of the healthcare graph data object; and
determine the one or more resemblance metrics based at least in part on the one or more cosine similarity measures.

9. The apparatus of claim 7, wherein, when calculating the one or more resemblance metrics, the at least one non-transitory memory and the computer program code are configured to, with the at least one processor, cause the apparatus to:
train the at least one graph-based machine learning model based at least in part on one or more training healthcare graph data objects and one or more training resemblance metrics, wherein each of the one or more training healthcare graph data objects comprises a plurality of training member subgraphs;
subsequent to training the at least one graph-based machine learning model, input the healthcare graph data object to the at least one graph-based machine learning model; and
receive, from the at least one graph-based machine learning model, the one or more resemblance metrics, wherein each of the one or more resemblance metrics indicates a similarity measure between one of the one or more historical member vertices and the member vertex.

10. The apparatus of claim 1, wherein a historical member query vertex of the one or more historical member query vertices is connected to a historical member vertex of the one or more historical member vertices in the healthcare graph data object via one or more edges, wherein the at least one non-transitory memory and the computer program code are configured to, with the at least one processor, cause the apparatus to:
determine, for the historical member query vertex, a member-query edge weight associated with the one or more edges; and
determine, for the historical member query vertex, a prioritization metrics of the one or more prioritization metrics based at least in part on the member-query edge weight.

11. The apparatus of claim 10, wherein, when determining the member-query edge weight, the at least one non-transitory memory and the computer program code are configured to, with the at least one processor, cause the apparatus to:
determine a healthcare outcome measure associated with the historical member vertex; and
calculate the member-query edge weight based at least in part on the healthcare outcome measure.

12. The apparatus of claim 10, wherein, when determining the member-query edge weight, the at least one non-transitory memory and the computer program code are configured to, with the at least one processor, cause the apparatus to:
determine a healthcare cost measure associated with the historical member vertex; and
calculate the member-query edge weight based at least in part on the healthcare cost measure.

13. The apparatus of claim 10, wherein the healthcare graph data object comprises a plurality of historical visit vertices, wherein a historical visit vertex of the plurality of historical visit vertices is connected to the historical member vertex and the historical member query vertex.

14. The apparatus of claim 13, wherein the one or more edges comprise a member-visit edge connecting the historical member vertex to the historical visit vertex and a visit-query edge connecting the historical visit vertex to the historical member query vertex.

15. The apparatus of claim 14, wherein the member-query edge weight is a combination of a member-visit edge weight associated with the member-visit edge and a visit-query edge weight associated with the visit-query edge.

16. The apparatus of claim 1, wherein, when performing the one or more prediction-based actions based at least in part on the one or more predicted member query vertices, the at least one non-transitory memory and the computer program code are configured to, with the at least one processor, cause the apparatus to:
render, on a graphic user interface displayed on a client computing entity, one or more predicted member query indications based at least in part on the one or more predicted member query vertices;
subsequent to rendering the one or more predicted member query indications, receive a user input associated with a predicted member query indication of the one or more predicted member query indications that corresponds to a predicted member query vertex of the one or more predicted member query vertices; and
determine a historical member query vertex corresponds to the predicted member query vertex.

17. The apparatus of claim 16, wherein the at least one non-transitory memory and the computer program code are configured to, with the at least one processor, cause the apparatus to:
determine that the user input indicates a user interest of the predicted member query indication; and
in response to determining that the user input indicates the user interest, increase a member-query edge weight of a member-query edge associated with the historical member query vertex.

18. The apparatus of claim 16, wherein the at least one non-transitory memory and the computer program code are configured to, with the at least one processor, cause the apparatus to:
determine that the user input indicates a user disinterest of the predicted member query indication; and
in response to determining that the user input indicates the user disinterest, decrease a member-query edge weight of a member-query edge associated with the historical member query vertex.

19. A computer-implemented method comprising:
generating one or more edges connecting one or more attribute vertices associated with a member vertex in a healthcare graph data object based at least in part on one or more member data objects comprising at least one of a member demographics data object, a member history data object, or a member symptom data object;
determining, using at least one graph-based machine learning model and based at least in part on the one or more attribute vertices, one or more historical member vertices from the healthcare graph data object at least according to one or more resemblance metrics associated with the one or more historical member vertices and relative to the member vertex;
determining, using the at least one graph-based machine learning model and based at least in part on the one or more historical member vertices, one or more historical member query vertices from the healthcare graph data object at least according to one or more prioritization metrics associated with the one or more historical member query vertices;
generating, based at least in part on the one or more historical member query vertices, one or more predicted member query vertices in the healthcare graph data object; and
performing one or more prediction-based actions based at least in part on the one or more predicted member query vertices.

20. A computer program product comprising at least one non-transitory computer-readable storage medium having computer-readable program code portions stored therein, the computer-readable program code portions comprising an executable portion configured to:
generate one or more edges connecting one or more attribute vertices to a member vertex in a healthcare graph data object based at least in part on one or more member data objects comprising at least one of a member demographics data object, a member history data object, or a member symptom data object;
determine, using at least one graph-based machine learning model and based at least in part on the one or more attribute vertices, one or more historical member vertices from the healthcare graph data object at least according to one or more resemblance metrics associated with the one or more historical member vertices and relative to the member vertex;
determine, using the at least one graph-based machine learning model and based at least in part on the one or more historical member vertices, one or more historical member query vertices from the healthcare graph data object at least according to one or more prioritization metrics associated with the one or more historical member query vertices;
generate, based at least in part on the one or more historical member query vertices, one or more predicted member query vertices in the healthcare graph data object; and
perform one or more prediction-based actions based at least in part on the one or more predicted member query vertices.
